(19) United States
(12) Patent Application Publication
(10) Pub. No.: US 2025/0136770 A1
(43) Pub. Date: May 1, 2025

(54) SILK FIBROIN DERIVED BRUSH-LIKE POLYMERS

(71) Applicants: UNIVERSITY OF CONNECTICUT, Farmington, CT (US); CONNECTICUT CHILDREN'S MEDICAL CENTER, Hartford, CT (US)

(72) Inventors: Kelly Anne Burke, Sutton, MA (US); Courtney K. Rowe, West Hartford, MA (US); Douglas H. Adamson, Mansfield Center, CT (US)

(21) Appl. No.: 18/692,742

(22) PCT Filed: Sep. 16, 2022
(86) PCT No.: PCT/US2022/076620

§ 371 Date: Mar. 15, 2024

Related U.S. Application Data
(60) Provisional application No. 63/245,591, filed on Sep. 17, 2021.

(51) Int. Cl. C08J 5/18; A61K 47/60; A61K 47/64

(52) U.S. Cl. CPC C08J 5/18; A61K 47/60; A61K 47/64; C08J 2389/00; C08J 2433/10

(57) ABSTRACT

Materials, methods, and techniques herein relate to relate to silk protein films. More specifically, the instant disclosure relates to the functionalization and properties of silk protein films, which may be used for treatment applications, such as medical implant devices.

### RELATED APPLICATION(S)

This application claims priority to U.S. Provisional Patent Application No. 63/245,591 filed on Sep. 17, 2021, the entire contents of which are incorporated herein by reference.

### FEDERALLY SPONSORED RESEARCH

This invention was made with government support under P200A150330 and P200A180065 awarded by the Department of Education and CHE 2004072 awarded by the National Science Foundation (NSF). The government has certain rights in the invention.

### REFERENCE TO SEQUENCE LISTING

This application is filed with a Computer Readable Form of a Sequence Listing in accord with 37 C.F.R. § 1.821 (c). The text file submitted by EFS, “209670-9063-WO01_sequence_listing_15 Sep. 2022.txt,” was created on Sep. 15, 2022, contains 1 sequence, has a file size of 5.4 Kbytes, and is hereby incorporated by reference in its entirety.

### TECHNICAL FIELD

Materials, methods, and techniques herein relate to relate to silk protein films. More specifically, the instant disclosure relates to the functionalization and properties of silk protein films, which may be used for treatment applications, such as medical implant devices.

### INTRODUCTION

Silk from the domesticated silkworm, Bombyx mori, is a tough and versatile material that has been used as a cloth and sutures. In native silk fibers, the amino acid sequence of the primary structural component of the silk protein, fibroin, may allow for close packing and highly aligned molecules that imbue the silk with desirable mechanical properties, e.g., providing high tensile strength with ductility and toughness. The natural silk fiber may rival synthetic polymer fibers with regards to its combination of strength, extensibility, and toughness (Fu et al. Chem. Comm., 2009 (43): 6515-29).

### SUMMARY

In one aspect, a composition comprising a silk protein film enriched with a plurality of hydroxyl groups is disclosed. The silk protein may be silk fibroin. The silk protein may comprise an amino acid sequence consisting of SEQ ID. No. 1.

The silk protein film enriched with a plurality of hydroxyl groups may further comprise a brush-like polymer attached to the silk protein film. The brush-like polymer may be present on the silk protein film at a density of 1.0 chains/nm² to 3.0 chains/nm².

The brush-like polymer may comprise a plurality of repeating units. In some instances, the brush-like polymer may comprise a first block and a second block. The first block may comprise a first plurality of repeating units. The second block may comprise a second block of repeating units.

Each repeating unit may comprise a backbone and a side chain. Each backbone may be independently derived from monomers, the monomers comprising an acrylate monomer, a methacrylate monomer, a vinyl monomer, or a combination thereof. Each side chain may independently comprise a neutral pendant group, a positively charged pendant group, a negatively charged pendant group, or a combination thereof.

In some instances, each side chain may independently comprise a zwitterionic pendant group, a hydrophilic pendant group, a hydrophobic pendant group, a pendant group attached to an active agent, or a combination thereof.

The zwitterionic pendant group may comprise a phosphodiester group, an ammonium group, a sulfonate group, a sulfobetaine group, a carboxybetaine group, a sulfopyridinium betaine group, a phosphorylcholine group, a cysteine group, a sulfobetaine siloxane group, a sulfobetaine acrylamide group, or a combination thereof.

The hydrophilic pendant group may comprise a polyethylene glycol (PEG) group, a hydroxyl group, an ether group, an ester group, a carboxylic acid group, a sulfonate group, an aldehyde group, a ketone group, a thiol group, an amine group, a nitro group, an imine group, a nitrile group, a thioether group, an amide group, or a combination thereof. In some instances, the polyethylene glycol (PEG) group has a molecular weight of 20 g/mol to 2*10⁶ g/mol.

The hydrophobic group may comprise a C₁₋₂₀alkyl group, a C₁₋₂₀haloalkyl group, a C₃₋₆ cycloalkyl group, a C₆₋₉aryl group, a 6- to 9-membered heteroaryl, or a combination thereof.

The active agent may be a cell, a protein, a peptide, a nucleic acid, a nucleic acid analog, a nucleotide, an oligonucleotide, a peptide nucleic acid (PNA), an aptamer, an antibody, an antigen, an epitope, a hormone, a hormone antagonist, a growth factor, a recombinant growth factor, a cell attachment mediator, a cytokine, an enzyme, a small molecule, an antibiotic, an antimicrobial compound, a virus, an antiviral, or a combination thereof.

In another aspect, a method for preparing a brush-like silk protein film is disclosed. The method may comprise enriching a silk protein film with a plurality of hydroxyl groups. The silk protein may be silk fibroin. The silk protein may comprise an amino acid sequence consisting of SEQ ID. No. 1. The silk protein film may be essentially free of sericin. In some instances, enriching the silk protein film may comprise reacting the silk protein film with ethylene oxide. In other instances, enriching the silk protein film may comprise oxidizing the silk protein film. Oxidizing the silk protein film may comprise photocatalytic oxidation.

The method for preparing a brush-like silk protein film may further comprise functionalizing at least one of the hydroxyl groups with a reactive group. Functionalizing the silk protein film may comprise reacting the silk protein film with an initiator. The initiator may be α-bromoisobutyryl bromide (BIBB).

The method for preparing a brush-like silk protein film may further comprise synthesizing a brush-like polymer on at least one reactive group. Synthesizing the brush-like polymer may comprise polymerizing monomers. Polymerizing the monomers may comprise free radical polymerization, atom transfer radical polymerization of the monomers, or reversible addition-fragmentation chain transfer.

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways.

### BRIEF DESCRIPTION OF THE DRAWINGS

FIG. 1 shows a flow chart depicting general film processing methods and the corresponding material compositions.

FIGS. 2A-2B show Attenuated Total Reflectance-Fourier Transform Infrared) ATR-FTIR) spectra of the surfaces of the unmodified and modified silk films. Vertical dashed reference lines in FIG. 2A correspond to S═O stretching at 1040 cm⁻¹ and ester C═O stretching at 1735 cm⁻¹, and the dashed reference line in FIG. 2B corresponds to ester C═O stretching at 1735 cm⁻¹.

FIG. 2A shows the ATR-FTIR spectra of (i) as cast silk fibroin (SF) film (SF_{As Cast}), hydroxyl enriched SF films ((ii) SF_{EO}, (iii) SF_{OX},), and SF films attached to brush-like polymers having zwitterionic side chains ((iv) SF_{EO}-zwitter, and (v) SF_{OX}-zwitter). The vertical dashed reference line in FIG. 2A corresponds to S═O stretching at 1040 cm⁻¹.

FIG. 2B shows the ATR-FTIR spectra for an unmodified SF film ((i) (SF_{As Cast})), hydroxyl enriched SF films ((ii) SF_{EO}, (iii) SF_{OX}), and SF films attached to brush-like polymers having PEG side chains ((iv) SF_{EO}-PEG, (v) SF_{OX}-PEG). The vertical dashed reference line in FIG. 2B corresponds to ester C═O stretching at 1735 cm⁻¹.

FIG. 3 is a flow chart overview of the synthesis steps for modifying the surface of the SF films, showing (i) Ethylene oxide treatment of as cast silk fibroin (SF) films (SF As Cast) to result in SF_{EO}; (ii, a) oxidation of unmodified water vapor annealed SF films (SF_{WVA})) using ammonium persulfate [aqueous 15% w/v APS, 5 min, 25° C.] and (ii, b) hydrolysis of sulfonate groups to hydroxyl groups [dH₂O, 10 min, 25° C.] to result in SF_{OX}; (iii, a) initiator attachment to hydroxylated SF films [triethyl amine (NEt₃), bromoisobutyryl bromide (BIBB), chloroform, 4 h, 40° C.], (iii, b) ATRP of zwitterionic monomer [CuBr/(2,2′-Bipyridine) Bipy, 2-(methacryloyloxy)ethyldimethyl-(3-sulfopropyl) ammonium hydroxide (DMAPS), methanol/water, 12 h, 25° C.] and (iii, c) ATRP of poly(ethylene glycol) methacrylate (PEGMA) monomer [CuBr/Bipy, PEGMA, methanol/water, 12 h, 25° C.]. Reaction (ii) has the potential to modify the amide backbone as well as numerous amino acid residues, therefore this scheme is not drawn to show the individual reactive sites that are modified.

FIGS. 4A-4B show water contact angles via the sessile drop method (FIG. 4A) and captive bubble method (FIG. 4B) of unmodified silk, hydroxylated silks, and silk brush films. “EO” refers to hydroxyl enrichment by ethylene oxide, and “OX” refers to hydroxyl enrichment by photo-catalyzed oxidation. The unmodified SF in “EO” series is “As Cast” (SF_{As Cast}), and unmodified SF in the “OX” series was water vapor annealed (SF_{WVA}) to induce beta sheet structures. For 4A-4B, * denotes significance at p<0.05 and # denotes not significant when compared to the unmodified SF within same synthesis group.

FIG. 4A is a bar graph showing contact angles of the SF film compositions via the sessile drop method.

FIG. 4B is a bar graph showing contact angles of the SF film compositions via the captive bubble method.

FIGS. 5A-5F show the atomic force microscopy (AFM) height micrographs of SF_{EO} (FIG. 5A), SF_{EO}-zwitter (FIG. 5B), SF_{EO}-PEG (FIG. 5C), SF_{OX} (FIG. 5D), SF_{OX}-zwitter (FIG. 5E), and SF_{OX}-PEG film surfaces (FIG. 5F).

FIG. 5A is the AFM height micrograph of a SF_{EO} film surface.

FIG. 5B is the AFM height micrograph of a SF_{EO}-zwitter film surface.

FIG. 5C is the AFM height micrograph of a SF_{EO}-PEG film surface.

FIG. 5D is the AFM height micrograph of a SF_{OX} film surface.

FIG. 5E is the AFM height micrograph of a SF_{OX}-zwitter film surface.

FIG. 5F is the AFM height micrograph of a SF_{OX}-PEG film surface.

FIGS. 6A-6B show the degradation profiles of unmodified water annealed silk (SF_{WVA}), hydroxylated silk (SF_{OX}), and the silk brush (SF_{OX}-zwitter and SF_{OX}-PEG) films in 1 U/mL protease XIV (FIG. 6A) and 1×PBS (FIG. 6B), at 37° C.

FIG. 6A shows the degradation profile of water annealed silk (SF_{WVA}), hydroxylated silk (SF_{OX}), and the silk brush (SF_{OX}-zwitter and SF_{OX}-PEG) films in 1 U/mL protease XIV.

FIG. 6B shows the degradation profile of water annealed silk (SF_{WVA}), hydroxylated silk (SF_{OX}), and the silk brush (SF_{OX}-zwitter and SF_{OX}-PEG) films in 1×PBS.

FIG. 7 shows amount (ng/cm²) of bovine serum albumin (BSA) adsorbed onto unmodified water vapor annealed silk (SF_{WVA}), oxidized silk (SF_{OX}), and silk brush-like polymer (SF_{OX}-zwitter, SF_{OX}-PEG, and SF-PEG_{CC}) film surfaces normalized to film area. * denotes significance at p<0.05.

FIGS. 8A-8B show the attachment (cells/cm²) of human mesenchymal stem cells (hMSCs) to unmodified water vapor annealed silk (SF_{WVA}), oxidized silk (SF_{OX}), and silk brush-like polymer (SF_{OX}-zwitter and SF_{OX}-PEG) film surfaces normalized to film area at 1 h, 3 h, 6 h, and 24 hours (FIG. 8A); and 24 hours (FIG. 8B). * denotes significance at p<0.05.

FIG. 8A is a graph of the attachment (cells/cm²) of human mesenchymal stem cells (hMSCs) to unmodified water vapor annealed silk (SF_{WVA}), oxidized silk (SF_{OX}), and silk brush-like polymer (SF_{OX}-zwitter and SF_{OX}-PEG) film surfaces normalized to film area at 1 h, 3 h, 6 h, and 24 h.

FIG. 8B is a bar graph showing the attachment (cells/cm²) of human mesenchymal stem cells (hMSCs) to unmodified water vapor annealed silk (SF_{WVA}), oxidized silk (SF_{OX}), and silk brush-like polymer (SF_{OX}-zwitter and SF_{OX}-PEG) film surfaces normalized to film area at 24 h.

FIG. 9 shows ATR-FTIR spectra of modified SF films (SF_{EO}, SF_{EO}-Br, and SF_{EO}-zwitter) that were initially treated with methanol. Reactive sites were enriched using EO treatment. Reference line shown at ˜1730 cm⁻¹.

FIG. 10 shows scanning electron microscopy-energy-dispersive X-ray spectroscopy (SEM-EDS) elemental analysis of SF_{OX}-Br.

FIG. 11 shows the ATR-FTIR spectra of modified SF films (SF_{OX}, SF_{OX}-Br, and SF_{OX}-zwitter) that were treated with methanol annealing (instead of water vapor annealing) before the film was enriched using the oxidative route (OX). Reference line shown at ˜1730 cm⁻¹.

FIG. 12 shows the AFM height micrograph of SF_{EO}-zwitter. This film was initially treated with methanol (instead of water vapor annealing) and the film was enriched using ethylene oxide (EO).

FIGS. 13A-13H show images of live cells and fluorescence intensity plots for the entire image area for unmodified SF_{WVA} (FIG. 13A, FIG. 13B), SF_{OX} (FIG. 13C, FIG. 13D), SF_{OX}-zwitter (FIG. 13E, FIG. 13F), SF_{OX}-PEG (FIG. 13G, FIG. 13H). Scale bars represent 100 μm.

FIG. 13A is a live cell image for SF_{WVA}.

FIG. 13B is a fluorescence intensity plot for SF_{WVA}.

FIG. 13C is a live cell image for SF_{OX}.

FIG. 13D is a fluorescence intensity plot for SF_{OX}.

FIG. 13E is a live cell image for SF_{OX}-zwitter.

FIG. 13F is a fluorescence intensity plot for SF_{OX}-zwitter.

FIG. 13G is a live cell image for SF_{OX}-PEG.

FIG. 13H is a fluorescence intensity plot for SF_{OX}-PEG.

FIGS. 14A-14B show “grafting to” and “grafting from” strategies to functionalize film surfaces.

FIG. 14A schematically illustrates the “grafting to” strategy for functionalizing film surfaces.

FIG. 14B schematically illustrates the “grafting from” strategy for functionalizing film surfaces.

FIGS. 15A-15B are general schematics that illustrate the versatility of the brush-like polymer SF film surfaces for therapeutic delivery applications.

FIG. 15A schematically illustrates therapeutic agent loading onto the brush-like polymer using a tethering molecule.

FIG. 15B schematically illustrates hydrolysis of the tethering molecule to release the therapeutic agent from the brush-like polymer.

FIGS. 16A-16B are general schematics showing the modulation of the brush-like polymer density on the surface of the SF films, the molecular weight of the brush-like polymer, density of the brush-like polymers on the films, the chemical composition of the brush-like polymer, the chemical composition of the tethering molecule, the length of the tethering molecule, and the type and the amount of therapeutic/therapeutics attached to the brush-like polymer.

FIG. 16A shows exemplary brush-like polymer SF films having various side chains to tune hydrophilicity.

FIG. 16B depicts exemplary brush-like polymer SF films having various tethers.

FIG. 16C shows exemplary brush-like polymer SF films having various amounts of reactive sidechain.

FIG. 17 schematically illustrates that exemplary silk surfaces may contain a combination of brush-like polymers arranged either next to each other (horizontal arrangement) or stacked brush-like polymers (vertical arrangement).

FIG. 18 schematically illustrates the processing of B. mori cocoons into pure silk fibroin (SF) solution that is then cast into biocompatible and biodegradable SF films. The image of film was taken in front of logo to demonstrate the film's transparency.

FIG. 19 schematically shows the synthesis of brush-like polymer SF films having hydrophobic blocks (Silk-MMA), hydrophilic blocks (Silk-DMAPS), and hydrophobic followed by hydrophilic blocks (Silk-MMA-DMAPS). DMAPS=2-(methacryloyloxy)ethyldimethyl-(3-sulfopropyl) ammonium hydroxide; and MMA=methylmethacrylate.

FIG. 20 is an ATR-FTIR spectrum demonstrating the successful polymerization of brush-like polymer blocks having varying hydrophilicities (order of hydrophilicity: DMAPS>AEMA>MMA) with vertical lines indicating stretching of ester (from repeat units) and sulfonate (from DMAPS).

FIG. 21 is a bar graph for water contact angle (°) showing that for SF films, the silk films become more hydrophobic with MMA blocks and more hydrophilic (less hydrophobic) with DMAPS blocks. Each block contains at least 20% AEMA for drug linking. Brush-like polymers with two blocks (MMA proximal and DMAPS distal) attached to the film, are hydrophilic. DMAPS=2-(methacryloyloxy)ethyldimethyl-(3-sulfopropyl) ammonium hydroxide; MMA=methylmethacrylate; and AEMA=2-aminoethylmethacrylate.

FIG. 22 is a graph showing that in vitro degradation of films containing 1 U/mL collagenase showed <20% mass remaining at 12 weeks. If collagenase is not used, films retain >80% of original mass remaining over this period.

FIG. 23 is a graph demonstrating that in vitro testing revealed no difference in fibroblast viability when cultured with medium incubated with silk and silk-brush films compared to control medium that was not incubated with films.

FIG. 24A is a graph showing the sustained release of bupivacaine from hydrophilic, hydrophobic, and intermediate block brush-like polymers. In contrast, the control non-block brush-like polymer SF films soaked (not covalently linked) with the drug, release all drug within 4 hours.

FIG. 24B is a graph showing sustained release of bupivacaine from brush-like polymer blocks of intermediate hydrophilicity at different loading levels.

FIG. 25 is a graph demonstrating the long-term compatibility of SF brush-like polymer films with 3T3 fibroblast cells. WVA=water vapor annealed (control); AEMA=2-aminoethylmethacrylate; MMA methylmethacrylate; DMAPS 2-(methacryloyloxy)ethyldimethyl-(3-sulfopropyl) ammonium hydroxide; and HEMA=(Hydroxyethyl) methacrylate.

### DETAILED DESCRIPTION

Exemplary materials, methods and techniques disclosed and contemplated herein generally relate to silk protein films. Silk protein films disclosed herein comprise a brush-like polymer, the brush-like polymer comprising repeating units, wherein each repeating unit comprises a backbone and a sidechain.

### I. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “may,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to a group —O-alkyl. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkoxyalkyl,” as used herein, refers to an alkoxy group, as defined herein, appended to the parent molecular moiety through an alkyl group, as defined herein.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl,” as used herein, means at least one amino group, as defined herein, is appended to the parent molecular moiety through an alkylene group, as defined herein.

The term “amino,” as used herein, means —NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cyanoalkyl,” as used herein, means at least one-CN group, is appended to the parent molecular moiety through an alkylene group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl). Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The terms cycloalkylene and heterocyclylene refer to divalent groups derived from the base ring, i.e., cycloalkane, heterocycle. For purposes of illustration, examples of cycloalkylene and heterocyclylene include, respectively,

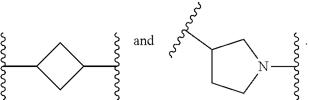 [SMILES: *C1CC(*)C1 ; *C1CCN(*)C1]

Cycloalkylene and heterocyclylene include a geminal divalent groups such as 1,1-C₃₋₆cycloalkylene (i.e.,

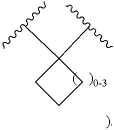

A further example is 1,1-cyclopropylene (i.e.,

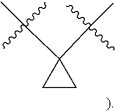 [SMILES: *C1(*)CC1]

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g., 1, 2, 3, or 4 heteroatoms independently selected from O, S, and N). The five membered aromatic monocyclic rings have two double bonds, and the six membered aromatic monocyclic rings have three double bonds. The bicyclic heteroaryl is an 8- to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro [3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, 3-oxaspiro[5.5]undecanyl, 6-oxaspiro[2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, COOH, ketone, amide, carbamate, and acyl.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

### II. Exemplary Compositions

Exemplary compositions described herein comprise a silk protein film enriched with a plurality of hydroxyl groups and a brush-like polymer attached to the silk protein film.

Silk fibroin is a particularly appealing protein polymer candidate to be used for various embodiments described herein, due to its versatile processing e.g., all-aqueous processing (Sofia et al., 54 J. Biomed. Mater. Res. 139 (2001); Perry et al. 20 Adv. Mater. 3070-72 (2008)), relatively easy functionalization (Murphy et al. 29 Biomat. 2829-38 (2008)), and biocompatibility (Santin et al. 46 J. Biomed. Mater. Res. 382-9 (1999)). For example, silk has been approved by U.S. Food and Drug Administration as a tissue engineering scaffold in human implants. See Altman et al., 24 Biomaterials: 401 (2003).

As used herein, the term “silk fibroin” or “fibroin” includes silkworm fibroin and insect or spider silk protein. See, e.g., Lucas et al. 13 Adv. Protein Chem. 107 (1958). Any type of silk fibroin may be used according to aspects of the present disclosure. Silk fibroin produced by silkworms, such as Bombyx mori, is the most common and represents an earth-friendly, renewable resource. For instance, silk fibroin may be attained by extracting sericin from the cocoons of B. mori. Organic silkworm cocoons are also commercially available. There are many different silks, however, including spider silk (e.g., obtained from Nephila clavipes), transgenic silks, genetically engineered silks (recombinant silk), such as silks from bacteria, yeast, mammalian cells, transgenic animals, or transgenic plants, and variants thereof, that may be used. See, e.g., WO 97/08315 and U.S. Pat. No. 5,245,012. In some instances, silk fibroin may be derived from other sources such as spiders, other silkworms, bees, and bioengineered variants thereof. In other instances, silk fibroin may be extracted from a gland of silkworm or transgenic silkworms. See, e.g., WO2007/098951. In various instances, silk fibroin is free, or essentially free of sericin, i.e., silk fibroin is a substantially sericin-depleted silk fibroin. The phrases “essentially free of sericin,” and “substantially sericin-depleted” may be used interchangeably to mean that no sericin is present or that sericin is present at an amount no greater than 0.0001 wt %.

Disclosed herein are silk protein films comprising a surface layer of brush-like polymers, wherein the brush-like polymers comprise repeat units modified by various pendant groups to increase attachment to silk surfaces.

As used herein, a “brush-like polymer” is a polymer chain tethered by one chain end to a solid interface (e.g., a silk fibroin film). A silk protein film comprising a brush-like polymer, as described herein, may be referred to as a “silk-brush film.”

Exemplary brush-like polymers may be present on the silk protein film at various densities, such as, without limitation, 1.0 chains/nm² to 3.0 chains/nm².

In various instances, the brush-like polymer is present at a density of 1.1 chains/nm² to 1.9 chains/nm²; 1.2 chains/nm² to 2.8 chains/nm²; 1.3 chains/nm² to 2.7 chains/nm²; 1.4 chains/nm² to 2.6 chains/nm²; 1.5 chains/nm² to 2.5 chains/nm²; 1.6 chains/nm² to 2.4 chains/nm²; 1.7 chains/nm² to 2.3 chains/nm²; 1.8 chains/nm² to 2.2 chains/nm²; or 1.9 chains/nm² to 2.1 chains/nm². In various instances, the brush-like polymer is present at a density of no greater than 3.0 chains/nm²; no greater than 2.9 chains/nm²; no greater than 2.8 chains/nm²; no greater than 2.7 chains/nm²; no greater than 2.6 chains/nm²; no greater than 2.5 chains/nm²; no greater than 2.4 chains/nm²; no greater than 2.3 chains/nm²; no greater than 2.2 chains/nm²; no greater than 2.1 chains/nm²; no greater than 2.0 chains/nm²; no greater than 2.0 chains/nm²; no greater than 1.9 chains/nm²; no greater than 1.8 chains/nm²; no greater than 1.7 chains/nm²; no greater than 1.6 chains/nm²; no greater than 1.5 chains/nm²; no greater than 1.4 chains/nm²; no greater than 1.3 chains/nm²; no greater than 1.2 chains/nm²; no greater than 1.1 chains/nm²; or no greater than 1.0 chains/nm². In various instances, the brush-like polymer is present at a density of no less than 1.0 chains/nm²; no less than 1.1 chains/nm²; no less than 1.2 chains/nm²; no less than 1.3 chains/nm²; no less than 1.4 chains/nm²; no less than 1.5 chains/nm²; no less than 1.6 chains/nm²; no less than 1.7 chains/nm²; no less than 1.8 chains/nm²; no less than 1.9 chains/nm²; no less than 2.0 chains/nm²; no less than 2.1 chains/nm²; no less than 2.2 chains/nm²; no less than 2.3 chains/nm²; no less than 2.4 chains/nm²; no less than 2.5 chains/nm²; no less than 2.6 chains/nm²; no less than 2.7 chains/nm²; no less than 2.8 chains/nm²; no less than 2.9 chains/nm²; or no less than 3.0 chains/nm².

Exemplary brush-like polymers have a plurality of repeating units, wherein each repeating unit comprises a backbone and a side chain. Each backbone may be independently derived from monomers. Exemplary monomers include, without limitation, an acrylate monomer, a methacrylate monomer, a vinyl monomer, and combinations thereof. Each side chain may independently comprise a neutral pendant group, a positively charged pendant group, a negatively charged pendant group, or a combination thereof.

As used herein, a “pendant group” or “side group” or side group is a molecule attached to a backbone chain of a long molecule.

In various instances, the pendant group may be a zwitterionic pendant group, a hydrophilic pendant group, a hydrophobic pendant group, a pendant group attached to an active agent, or a combination thereof.

Exemplary zwitterionic pendant groups include, without limitation, phosphodiester groups, ammonium groups, sulfonate groups, sulfobetaine groups, carboxybetaine groups, sulfopyridinium betaine groups, phosphorylcholine groups, cysteine groups, sulfobetaine siloxane groups, sulfobetaine acrylamide groups, and combinations thereof.

Exemplary hydrophilic pendant groups include, without limitation, polyethylene glycol (PEG) groups, hydroxyl groups, ether groups, ester groups, carboxylic acid groups, sulfonate groups, aldehyde groups, ketone groups, thiol groups, amine groups, nitro groups, imine groups, nitrile groups, thioether groups, amide groups, and combinations thereof.

In some instances, the brush-like polymers are modified with a polyethylene glycol (PEG) pendant group. The PEG group may be incorporated to reduce biofouling. As used herein, the term “polyethylene glycol” or “PEG” means an ethylene glycol polymer that contains about 20 to about 2000000 linked monomers, typically about 50 to about 1000 linked monomers. PEG is also known as polyethylene oxide (PEO) or polyoxyethylene (POE), depending on its molecular weight. Generally, PEG, PEO, and POE are chemically synonymous, but PEG has previously tended to refer to oligomers and polymers with a molecular mass below 20,000 g/mol, PEO to polymers with a molecular mass above 20,000 g/mol, and POE to a polymer of any molecular mass. PEG and PEO are liquids or low-melting solids, depending on their molecular weights.

PEGs are prepared by polymerization of ethylene oxide and are commercially available over a wide range of molecular weights from 300 g/mol to 10,000,000 g/mol. In various instances, the polyethylene glycol (PEG) may have a molecular weight (MW) of 44 to 2*10⁶ g/mol. In various instances, the polyethylene glycol (PEG) may have a molecular weight (MW) of 44 to 2*10⁵ g/mol; 200 to 2*10⁴ g/mol; or 200 to 2,000 g/mol. In various instances, the polyethylene glycol (PEG) may have a molecular weight (MW) of no greater than 2*10⁶ g/mol; no greater than 2*10⁵ g/mol; no greater than 2*10⁴ g/mol; no greater than 2,000 g/mol; no greater than 200 g/mol; or no greater than 44 g/mol. In various instances, the polyethylene glycol (PEG) may have a molecular weight (MW) of no less than 44 g/mol; no less than 200 g/mol; no less than 2000 g/mol; no less than 2*10⁴ g/mol; no less than 2*10⁵ g/mol; or no less than 2*10⁶ g/mol.

While PEG and PEO with different molecular weights find use in different applications, and have different physical properties (e.g., viscosity) due to chain length effects, their chemical properties are nearly identical. Different forms of PEG are also available, depending on the initiator used for the polymerization process—the most common initiator is a monofunctional methyl ether PEG, or methoxypoly(ethylene glycol), abbreviated mPEG. Lower-molecular-weight PEGs are also available as purer oligomers, referred to as monodisperse, uniform, or discrete PEGs are also available with different geometries.

As used herein, the term PEG is intended to be inclusive and not exclusive. The term PEG includes polyethylene glycol in any of its forms, including alkoxy PEG, difunctional PEG, multiarmed PEG, forked PEG, branched PEG, pendent PEG (i.e., PEG or related polymers having one or more functional groups pendent to the polymer backbone), or PEG With degradable linkages therein. Further, the PEG backbone may be linear or branched. Branched polymer backbones are generally known in the art. Typically, a branched polymer has a central branch core moiety and a plurality of linear polymer chains linked to the central branch core. PEG is commonly used in branched forms that may be prepared by addition of ethylene oxide to various polyols, such as glycerol, pentaerythritol and sorbitol. The central branch moiety may also be derived from several amino acids, such as lysine. The branched polyethylene glycol may be represented in general form as R(-PEG-OH)ₘ in which R represents the core moiety, such as glycerol or pentaerythritol, and m represents the number of arms. Multi-armed PEG molecules, such as those described in U.S. Pat. No. 5,932,462, may also be used as biocompatible polymers.

Exemplary PEGs include, but are not limited to, PEG20, PEG30, PEG40, PEG60, PEG80, PEG100, PEG115, PEG200, PEG 300, PEG400, PEG500, PEG600, PEG750, PEG1000, PEG1500, PEG2000, PEG3350, PEG4000, PEG4600, PEG5000, PEG6000, PEG8000, PEG11000, PEG12000, PEG15000, PEG 20000, PEG250000, PEG500000, PEG100000, PEG2000000 and the like.

Exemplary hydrophobic groups include, without limitation, C₁₋₂₀alkyl groups, C₁₋₂₀haloalkyl groups, C₃₋₆cycloalkyl groups, C₆₋₁₂aryl groups, and combinations thereof.

Examples of active agent(s) include, without limitation, a therapeutic agent, or a biological material, such as cells (including stem cells such as induced pluripotent stem cells), proteins, peptides, nucleic acids (e.g., DNA, RNA, siRNA), nucleic acid analogs, nucleotides, oligonucleotides, peptide nucleic acids (PNA), aptamers, antibodies or fragments or portions thereof (e.g., paratopes or complementarity-determining regions), antigens or epitopes, hormones, hormone antagonists, growth factors or recombinant growth factors and fragments and variants thereof, cell attachment mediators (such as RGD), cytokines, enzymes, small molecules, antibiotics or antimicrobial compounds, viruses, antivirals, toxins, therapeutic agents and prodrugs, small molecules and any combinations thereof. See, e.g., WO 2009/140588; U.S. Patent Application Ser. No. 61/224,618). The active agent may also be a combination of any of the above-mentioned agents. Encapsulating either a therapeutic agent or biological material, or the combination of them, is desirous because the encapsulated composition may be used for numerous biomedical purposes.

In various instances, the active agent may also be an organism such as a fungus, plant, animal, bacterium, or a virus (including bacteriophage). Moreover, the active agent may include neurotransmitters, hormones, intracellular signal transduction agents, pharmaceutically active agents, toxic agents, agricultural chemicals, chemical toxins, biological toxins, microbes, and animal cells such as neurons, liver cells, and immune system cells. The active agents may also include therapeutic compounds, such as pharmacological materials, vitamins, sedatives, hypnotics, prostaglandins, and radiopharmaceuticals.

Exemplary cells suitable for use herein may include, but are not limited to, progenitor cells or stem cells (including, e.g., induced pluripotent stem cells), smooth muscle cells, skeletal muscle cells, cardiac muscle cells, epithelial cells, endothelial cells, urothelial cells, fibroblasts, myoblasts, ocular cells, chondrocytes, chondroblasts, osteoblasts, osteoclasts, keratinocytes, kidney tubular cells, kidney basement membrane cells, integumentary cells, bone marrow cells, hepatocytes, bile duct cells, pancreatic islet cells, thyroid, parathyroid, adrenal, hypothalamic, pituitary, ovarian, testicular, salivary gland cells, adipocytes, and precursor cells. The active agents may also be the combinations of any of the cells listed above. See also WO 2008/106485; WO 2010/040129; WO 2007/103442.

As used herein, the terms “proteins” and “peptides” are used interchangeably herein to designate a series of amino acid residues connected to the other by peptide bonds between the alpha-amino and carboxy groups of adjacent residues. The terms “protein”, and “peptide”, which are used interchangeably herein, refer to a polymer of protein amino acids, including modified amino acids (e.g., phosphorylated, glycated, etc.) and amino acid analogs, regardless of its size or function. Although “protein” is often used in reference to relatively large polypeptides, and “peptide” is often used in reference to small polypeptides, usage of these terms in the art overlaps and varies. The term “peptide” as used herein refers to peptides, polypeptides, proteins, and fragments of proteins, unless otherwise noted. The terms “protein” and “peptide” are used interchangeably herein when referring to a gene product and fragments thereof. Thus, exemplary peptides or proteins include gene products, naturally occurring proteins, homologs, orthologs, paralogs, fragments and other equivalents, variants, fragments, and analogs of the foregoing.

The term “nucleic acids” used herein refers to polynucleotides such as deoxyribonucleic acid (DNA), and, where appropriate, ribonucleic acid (RNA), polymers thereof in either single- or double-stranded form. Unless specifically limited, the term encompasses nucleic acids containing known analogs of natural nucleotides, which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer, et al., Nucleic Acid Res. 19:5081 (1991); Ohtsuka, et al., J. Biol. Chem. 260:2605-2608 (1985), and Rossolini, et al., Mol. Cell. Probes 8:91-98 (1994)). The term “nucleic acid” should also be understood to include, as equivalents, derivatives, variants, and analogs of either RNA or DNA made from nucleotide analogs, and single (sense or antisense) and double-stranded polynucleotides. The term “nucleic acid” also encompasses modified RNA (modRNA). The term “nucleic acid” also encompasses siRNA, shRNA, or any combinations thereof.

The term “modified RNA” means that at least a portion of the RNA has been modified, e.g., in its ribose unit, in its nitrogenous base, in its internucleoside linkage group, or any combinations thereof. Accordingly, in various instances, a “modified RNA” may contain a sugar moiety which differs from ribose, such as a ribose monomer where the 2′—OH group has been modified. Alternatively, or in addition to being modified at its ribose unit, a “modified RNA” may contain a nitrogenous base which differs from A, C, G and U (a “non-RNA nucleobase”), such as T or MeC. In various instances, a “modified RNA” may contain an internucleoside linkage group which is different from phosphate (—O—P(O)₂—O—), such as —O—P(O,S)—O—. In various instances, a modified RNA may encompass locked nucleic acid (LNA).

The term “short interfering RNA” (siRNA), also referred to herein as “small interfering RNA” is defined as an agent which functions to inhibit expression of a target gene, e.g., by RNAi. An siRNA may be chemically synthesized, it may be produced by in vitro transcription, or it may be produced within a host cell. siRNA molecules may also be generated by cleavage of double stranded RNA, where one strand is identical to the message to be inactivated. The term “siRNA” refers to small inhibitory RNA duplexes that induce the RNA interference (RNAi) pathway. These molecules may vary in length (generally 18-30 base pairs) and contain varying degrees of complementarity to their target mRNA in the antisense strand. Some, but not all, siRNA have unpaired overhanging bases on the 5′ or 3′ end of the sense 60 strand and/or the antisense strand. The term “siRNA” includes duplexes of two separate strands, as well as single strands that may form hairpin structures comprising a duplex region.

The term “shRNA” as used herein refers to short hairpin RNA which functions as RNAi and/or siRNA species but differs in that shRNA species are double stranded hairpin-like structure for increased stability. The term “RNAi” as used herein refers to interfering RNA, or RNA interference molecules are nucleic acid molecules or analogues thereof for example RNA-based molecules that inhibit gene expression. RNAi refers to a means of selective post-transcriptional gene silencing. RNAi may result in the destruction of specific mRNA, or prevents the processing or translation of RNA, such as mRNA.

The term “enzymes” as used here refers to a protein molecule that catalyzes chemical reactions of other substances without it being destroyed or substantially altered upon completion of the reactions. The term may include naturally occurring enzymes and bioengineered enzymes or mixtures thereof. Examples of enzyme families include, but are not limited to, peroxidase, lipase, amylose, organophosphate dehydrogenase, ligases, restriction endonucleases, ribonucleases, DNA polymerases, glucose oxidase, laccase, kinases, dehydrogenases, oxidoreductases, GTPases, carboxyl transferases, acyl transferases, decarboxylases, transaminases, racemases, methyl transferases, formyl transferases, and α-ketodecarboxylases.

As used herein, the term “aptamers” means a single-stranded, partially single-stranded, partially double-stranded, or double-stranded nucleotide sequence capable of specifically recognizing a selected non-oligonucleotide molecule or group of molecules. In various instances, the aptamer recognizes the non-oligonucleotide molecule or group of molecules by a mechanism other than Watson-Crick base pairing or triplex formation. Aptamers may include, without limitation, defined sequence segments and sequences comprising nucleotides, ribonucleotides, deoxyribonucleotides, nucleotide analogs, modified nucleotides and nucleotides comprising backbone modifications, branchpoints and non-nucleotide residues, groups, or bridges. Methods for selecting aptamers for binding to a molecule are widely known in the art and easily accessible to one of ordinary skill in the art.

As used herein, the term “antibody” or “antibodies” refers to an intact immunoglobulin or to a monoclonal or polyclonal antigen-binding fragment with the Fc (crystallizable fragment) region or FcRn binding fragment of the Fc region. The term “antibodies” also includes “antibody-like molecules”, such as fragments of the antibodies, e.g., antigen-binding fragments. Antigen-binding fragments may be produced by recombinant DNA techniques or by enzymatic or chemical cleavage of intact antibodies. “Antigen-binding fragments” include, inter alia, Fab, Fab′, F(ab′)2, Fv, dAb, and complementarity determining region (CDR) fragments, single-chain antibodies (scFv), single domain antibodies, chimeric antibodies, diabodies, and polypeptides that contain at least a portion of an immunoglobulin that is sufficient to confer specific antigen binding to the polypeptide. Linear antibodies are also included for the purposes described herein. The terms Fab, Fc, pFc′, F(ab′)2 and Fv are employed with standard immunological meanings (Klein, Immunology (John Wiley, New York, N.Y., 1982); Clark, W. R. (1986) The Experimental Foundations of Modern Immunology (Wiley & Sons, Inc., New York); and Roitt, I. (1991) Essential Immunology, 7th Ed., (Blackwell Scientific Publications, Oxford)). Antibodies or antigen-binding fragments specific for various antigens are available commercially from vendors such as R&D Systems, BD Biosciences, e-Biosciences and Miltenyi, or may be raised against these cell-surface markers by methods known to those skilled in the art.

Exemplary antibodies that may be incorporated in silk fibroin include, but are not limited to, abciximab, adalimumab, alemtuzumab, basiliximab, bevacizumab, cetuximab, certolizumab pegol, daclizumab, eculizumab, efalizumab, gemtuzumab, ibritumomab tiuxetan, infliximab, muromonab-CD3, natalizumab, ofatumumab omalizumab, palivizumab, panitumumab, ranibizumab, rituximab, tositumomab, trastuzumab, altumomab pentetate, arcitumomab, atlizumab, bectumomab, belimumab, besilesomab, biciromab, canakinumab, capromab pendetide, catumaxomab, denosumab, edrecolomab, efungumab, ertumaxomab, etaracizumab, fanolesomab, fontolizumab, gemtuzumab ozogamicin, golimumab, igovomab, imciromab, labetuzumab, mepolizumab, motavizumab, nimotuzumab, nofetumomab merpentan, oregovomab, pemtumomab, pertuzumab, rovelizumab, ruplizumab, sulesomab, tacatuzumab tetraxetan, tefibazumab, tocilizumab, ustekinumab, visilizumab, votumumab, zalutumumab, and zanolimumab. The active agents may also be the combinations of any of the antibodies listed above.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-17 (1987)). In some instances, a complementarity determining region may include amino acids from both a CDR region defined according to Kabat and a hypervariable loop.

The expression “linear antibodies” refers to the antibodies described in Zapata et al., Protein Eng., 8 (10): 1057-1062 (1995). Briefly, these antibodies comprise a pair of tandem Fd segments (VH-CH1-VH-CH1) which, together with complementary light chain polypeptides, form a pair of antigen binding regions. Linear antibodies may be bispecific or monospecific.

The expression “single-chain Fv” or “scFv” antibody fragments, as used herein, is intended to mean antibody fragments that comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. (The Pharmacology of Monoclonal Antibodies, vol. 113, Rosenburg and Moore eds., Springer-Verlag, New York, pp. 269-315 (1994)).

The term “diabodies,” as used herein, refers to small antibody fragments with two antigen-binding sites, which fragments comprise a heavy-chain variable domain (VH) Connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. (EP 404,097; WO 93/11161; Hollinger et ah, Proc. Natl. Acad. Sd. USA, PO: 6444-6448 (1993)).

As used herein, the term “small molecules” refers to natural or synthetic molecules including, but not limited to, peptides, peptidomimetics, amino acids, amino acid analogs, polynucleotides, polynucleotide analogs, aptamers, nucleotides, nucleotide analogs, organic or inorganic compounds (i.e., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “antibiotics” or “antimicrobial compound” is used herein to describe a compound or composition which decreases the viability of a microorganism, or which inhibits the growth or reproduction of a microorganism. As used in this disclosure, an antibiotic is further intended to include an antimicrobial, bacteriostatic, or bactericidal agent. Exemplary antibiotics may include, but are not limited to, actinomycin; aminoglycosides (e.g., neomycin, gentamicin, tobramycin); β-lactamase inhibitors (e.g., clavulanic acid, sulbactam); glycopeptides (e.g., vancomycin, teicoplanin, polymixin); ansamycins; bacitracin; carbacephem; carbapenems; cephalosporins (e.g., cefazolin, cefaclor, cefditoren, ceftobiprole, cefuroxime, cefotaxime, cefipeme, cefadroxil, cefoxitin, cefprozil, cefdinir); gramicidin; isoniazid; linezolid; macrolides (e.g., erythromycin, clarithromycin, azithromycin); mupirocin; penicillins (e.g., amoxicillin, ampicillin, cloxacillin, dicloxacillin, flucloxacillin, oxacillin, piperacillin); oxolinic acid; polypeptides (e.g., bacitracin, polymyxin B); quinolones (e.g., ciprofloxacin, nalidixic acid, enoxacin, gatifloxacin, levaquin, ofloxacin, etc.); sulfonamides (e.g., sulfasalazine, trimethoprim, trimethoprim-sulfamethoxazole (co-trimoxazole), sulfadiazine); tetracyclines (e.g., doxycyline, minocycline, tetracycline, etc.); monobactams such as aztreonam; chloramphenicol; lincomycin; clindamycin; ethambutol; mupirocin; metronidazole; pefloxacin; pyrazinamide; thiamphenicol; rifampicin; thiamphenicl; dapsone; clofazimine; quinupristin; metronidazole; linezolid; isoniazid; piracil; novobiocin; trimethoprim; fosfomycin; fusidic acid; or other topical antibiotics. Optionally, the antibiotic agents may also be antimicrobial peptides such as defensins, magainin and nisin; or lytic bacteriophage. The antibiotic agents may also be the combinations of any of the agents listed above. See also PCT/US2010/026190.

As used herein, the term “antigens” refers to a molecule or a portion of a molecule capable of being bound by a selective binding agent, such as an antibody, and additionally capable of being used in an animal to elicit the production of antibodies capable of binding to an epitope of that antigen. An antigen may have one or more epitopes. The term “antigen” may also refer to a molecule capable of being bound by an antibody or a T cell receptor (TCR) if presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant. An antigen may have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens.

As used herein, the term “therapeutic agent” generally means a molecule, group of molecules, complex or substance administered to an organism for diagnostic, therapeutic, preventative medical, or veterinary purposes. As used herein, the term “therapeutic agent” includes a “drug” or a “vaccine.” This term includes externally and internally administered topical, localized, and systemic human and animal pharmaceuticals, treatments, remedies, nutraceuticals, cosmeceuticals, biologicals, devices, diagnostics, and contraceptives, including preparations useful in clinical and veterinary screening, prevention, prophylaxis, healing, wellness, detection, imaging, diagnosis, therapy, surgery, monitoring, cosmetics, prosthetics, forensics, and the like. This term may also be used in reference to agricultural, workplace, military, industrial and environmental therapeutics, or remedies comprising selected molecules or selected nucleic acid sequences capable of recognizing cellular receptors, membrane receptors, hormone receptors, therapeutic receptors, microbes, viruses, or selected targets comprising or capable of contacting plants, animals and/or humans. This term may also specifically include nucleic acids and compounds comprising nucleic acids that produce a bioactive effect, for example deoxyribonucleic acid (DNA), ribonucleic acid (RNA), modified DNA or RNA, or mixtures or combinations thereof, including, for example, DNA nanoplexes.

The term “therapeutic agent” also includes an active agent that is capable of providing a local or systemic biological, physiological, or therapeutic effect in the biological system to which it is applied. For example, the therapeutic agent may act to control infection or inflammation, enhance cell growth and tissue regeneration, control tumor growth, act as an analgesic, promote anti-cell attachment, and enhance bone growth, among other functions. Other suitable therapeutic agents may include anti-viral agents, hormones, antibodies, or therapeutic proteins. Other therapeutic agents include prodrugs, which are agents that are not biologically active when administered but, upon administration to a subject are converted to biologically active agents through metabolism or an alternative mechanism. Additionally, a silk-based composition may contain combinations of two or more therapeutic agents.

In various instances, different types of therapeutic agents that may be encapsulated or dispersed in a silk fibroin-based material may include, but not limited to, proteins, peptides, antigens, immunogens, vaccines, antibodies, or portions thereof, antibody-like molecules, enzymes, nucleic acids, modified RNA, siRNA, shRNA, aptamers, small molecules, antibiotics, and any combinations thereof.

Exemplary therapeutic agents include, but are not limited to, those found in Harrison's Principles of Internal Medicine, 13th Edition, Eds. T. R. Harrison et al. McGraw-Hill N.Y., NY; Physicians Desk Reference, 50th Edition, 1997, Oradell N.J., Medical Economics Co.; Pharmacological Basis of Therapeutics, 8th Edition, Goodman and Gilman, 1990; United States Pharmacopeia, The National Formulary, USP XII NF XVII, 1990, the complete contents of all of which are incorporated herein by reference.

Therapeutic agents include the herein disclosed categories and specific examples. It is not intended that the category be limited by the specific examples. Those of ordinary skill in the art will recognize also numerous other compounds that fall within the categories and that are useful according to the present disclosure. Exemplary therapeutic agents, include, but are not limited to, a radiosensitizer, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, a vaccine, a protein, or a nucleic acid. In a further aspect, the pharmaceutically active agent may be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; anti-inflammatory agents, including antiasthmatic anti-inflammatory agents, antiarthritis anti-inflammatory agents, and non-steroidal anti-inflammatory agents, examples of which include but are not limited to sulfides, mesalamine, budesonide, salazopyrin, diclofenac, pharmaceutically acceptable diclofenac salts, nimesulide, naproxene, acetaminophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, bupivacaine, ropivacaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as Coumadin, warfarin, acetylsalicylic acid, and ticlopidine; sedatives such as benzodiazepines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; or nucleic acids such as DNA sequences encoding for biological proteins, and antisense oligonucleotides.

Exemplary anti-cancer agents include, but are not limited to, alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelin receptor antagonists, retinoic acid receptor agonists, immuno-modulators, hormonal and antihormonal agents, photodynamic agents, and tyrosine kinase inhibitors.

Exemplary antibiotics include, but are not limited to, aminoglycosides (e.g., gentamicin, tobramycin, netilmicin, streptomycin, amikacin, neomycin), bacitracin, corbapenems (e.g., imipenem/cislastatin), cephalosporins, colistin, methenamine, monobactams (e.g., aztreonam), penicillins (e.g., penicillin G, penicillinV, methicillin, natcillin, oxacillin, cloxacillin, dicloxacillin, ampicillin, amoxicillin, carbenicillin, ticarcillin, piperacillin, mezlocillin, azlocillin), polymyxin B, quinolones, and vancomycin; and bacteriostatic agents such as chloramphenicol, clindanyan, macrolides (e.g., erythromycin, azithromycin, clarithromycin), lincomyan, nitrofurantoin, sulfonamides, tetracyclines (e.g., tetracycline, doxycycline, minocycline, demeclocyline), and trimethoprim. Also included are metronidazole, fluoroquinolones, and ritampin.

Enzyme inhibitors are substances which inhibit an enzymatic reaction. Exemplary enzyme inhibitors include, but are not limited to, edrophonium chloride, N-methylphysostigmine, neostigmine bromide, physostigmine sulfate, tacrine, tacrine, 1-hydroxy maleate, iodotubercidin, p-bromotetramiisole, 10-(alpha-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylamine, N′-monomethyl-Larginine acetate, carbidopa, 3-hydroxybenzylhydrazine, hydralazine, clorgyline, deprenyl, hydroxylamine, iproniazid phosphate, 6-Me0-tetrahydro-9H-pyrido-indole, nialamide, pargyline, quinacrine, semicarbazide, tranylcypromine, N,N-diethylaminoethyl-2,2-diphenylvalerate hydrochloride, 3-isobutyl-1-methylxanthne, papaverine, indomethacind, 2-cyclooctyl-2-hydroxyethylamine hydrochloride, 2,3-dichloro-a-methylbenzylamine (DCMB), 8,9-dichloro-2,3,4,5-tetrahydro-1H-2-benzazepine hydrochloride, p-amino glutethimide, p-aminoglutethimide tartrate, 3-iodotyrosine, alpha-methyltyrosine, acetazolamide, dichlorphenamide, 6-hydroxy-2-benzothiazolesulfonamide, and allopurinol.

Exemplary antihistamines include, but are not limited to, pyrilamine, chlorpheniramine, and tetrahydrazoline.

Exemplary anti-inflammatory agents include, but are not limited to, corticosteroids, nonsteroidal anti-inflammatory drugs (e.g., aspirin, phenylbutazone, indomethacin, sulindac, tolmetin, ibuprofen, piroxicam, and fenamates), acetaminophen, phenacetin, gold salts, chloroquine, D-Penicillamine, methotrexate colchicine, allopurinol, probenecid, and sulfinpyrazone.

Exemplary muscle relaxants include, but are not limited to, mephenesin, methocarbomal, cyclobenzaprine hydrochloride, trihexylphenidyl hydrochloride, levodopa/carbidopa, and biperiden.

Exemplar anti-spasmodics include, but are not limited to, atropine, scopolamine, oxyphenonium, and papaverine.

Exemplary analgesics include, but are not limited to, aspirin, phenybutazone, idomethacin, sulindac, tolmetic, ibuprofen, piroxicam, fenamates, acetaminophen, phenacetin, morphine sulfate, codeine sulfate, meperidine, nalorphine, opioids (e.g., codeine sulfate, fentanyl citrate, hydrocodone bitartrate, loperamide, morphine sulfate, noscapine, norcodeine, normorphine, thebaine, nor-binaltorphimine, buprenorphine, chlomaltrexamine, funaltrexamione, nalbuphine, nalorphine, naloxone, naloxonazine, naltrexone, and naltrindole), procaine, lidocaine, tetracaine, bupivacaine, ropivacaine, and dibucaine.

Exemplary ophthalmic agents include, but are not limited to, sodium fluorescein, rose bengal, methacholine, adrenaline, cocaine, atropine, alpha-chymotrypsin, hyaluronidase, betaxalol, pilocarpine, timolol, timolol salts, and combinations thereof.

Prostaglandins are art recognized and are a class of naturally occurring chemically related, long-chain hydroxy fatty acids that have a variety of biological effects.

Anti-depressants are substances capable of preventing or relieving depression. Exemplary anti-depressants include, but are not limited to, imipramine, amitriptyline, nortriptyline, protriptyline, desipramine, amoxapine, doxepin, maprotiline, tranylcypromine, phenelzine, and isocarboxazide.

Trophic factors are factors whose continued presence improves the viability or longevity of a cell. Exemplary trophic factors include, but are not limited to, platelet-derived growth factor (PDGP), neutrophil-activating protein, monocyte chemoattractant protein, macrophage-inflammatory protein, platelet factor, platelet basic protein, and melanoma growth stimulating activity; epidermal growth factor, transforming growth factor (alpha), fibroblast growth factor, platelet-derived endothelial cell growth factor, insulin-like growth factor, glial derived growth neurotrophic factor, ciliary neurotrophic factor, nerve growth factor, bone growth/cartilage-inducing factor (alpha and beta), bone morphogenetic proteins, interleukins (e.g., interleukin inhibitors or interleukin receptors, including interleukin 1 through interleukin 10), interferons (e.g., interferon alpha, beta and gamma), hematopoietic factors, including erythropoietin, granulocyte colony stimulating factor, macrophage colony stimulating factor and granulocyte-macrophage colony stimulating factor; tumor necrosis factors, and transforming growth factors (beta), including beta-1, beta-2, beta-3, inhibin, and activin.

Exemplary hormones include, but are not limited to, estrogens (e.g., estradiol, estrone, estriol, diethylstibestrol, quinestrol, chlorotrianisene, ethinyl estradiol, mestranol), anti-estrogens (e.g., clomiphene, tamoxifen), progestins (e.g., medroxyprogesterone, norethindrone, hydroxyprogesterone, norgestrel), antiprogestin (mifepristone), androgens (e.g., testosterone cypionate, fluoxymesterone, danazol, testolactone), anti-androgens (e.g., cyproterone acetate, flutamide), thyroid hormones (e.g., triiodothyronne, thyroxine, propylthiouracil, methimazole, and iodixode), and pituitary hormones (e.g., corticotropin, sumutotropin, oxytocin, and vasopressin). Hormones are commonly employed in hormone replacement therapy and/or for purposes of birth control. Steroid hormones, such as prednisone, are also used as immunosuppressants and anti-inflammatories.

### III. Exemplary Methods for Preparing Brush-Like Silk Protein Films

Exemplary methods for preparing brush-like silk protein films described herein comprise enriching a silk protein film with a plurality of hydroxyl groups, functionalizing at least one of the hydroxyl groups with a reactive group, and synthesizing a brush-like polymer on at least one reactive group. In some instances, enriching the silk protein film with a plurality of hydroxyl groups comprises reacting the silk protein film with ethylene oxide. In other instances, enriching the silk protein film with a plurality of hydroxyl groups comprises oxidizing the silk protein film. Exemplary oxidation methods include, without limitation, photocatalytic oxidation. Exemplary methods of functionalizing the silk protein film comprises reacting the silk protein film with an initiator. An exemplary initiator includes, without limitation, α-bromoisobutyryl bromide (BIBB).

In some instances, synthesizing the brush-like polymer comprises polymerizing monomers. Exemplary methods for polymerizing the monomers comprise, without limitation, free radical polymerization, atom transfer radical polymerization of the monomers, and reversible addition-fragmentation chain transfer.

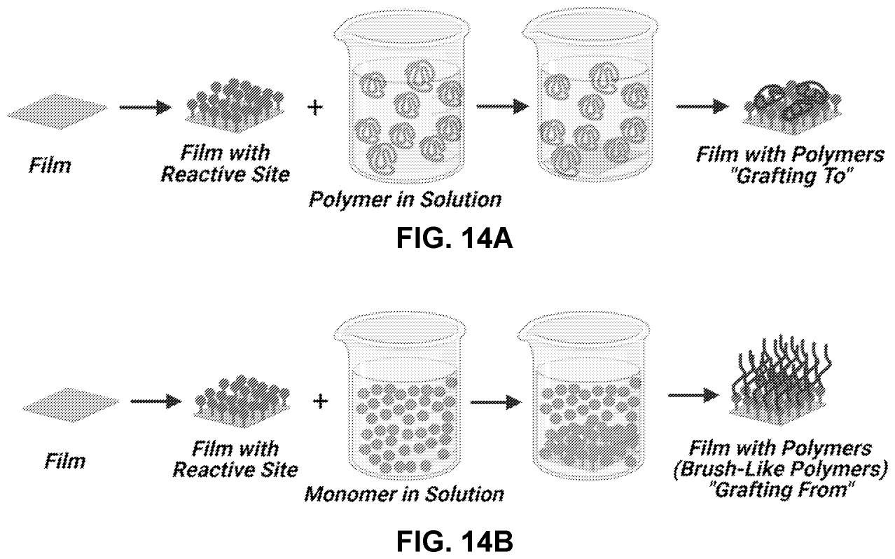

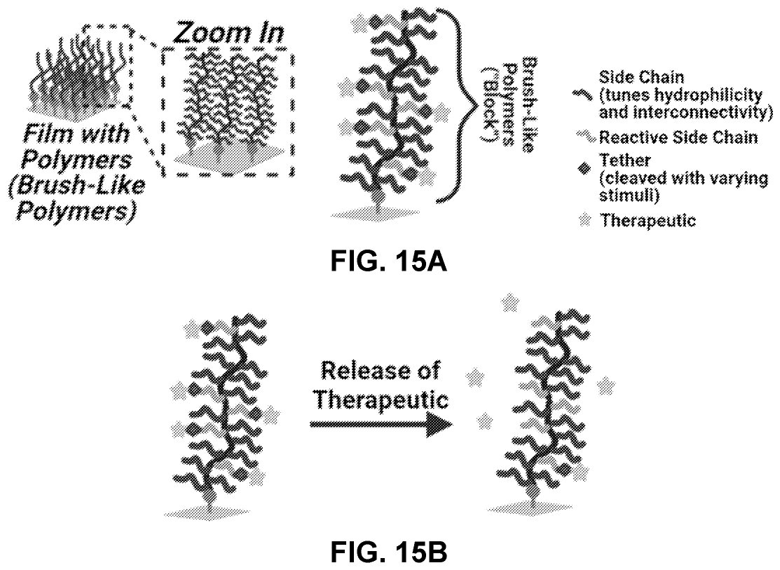

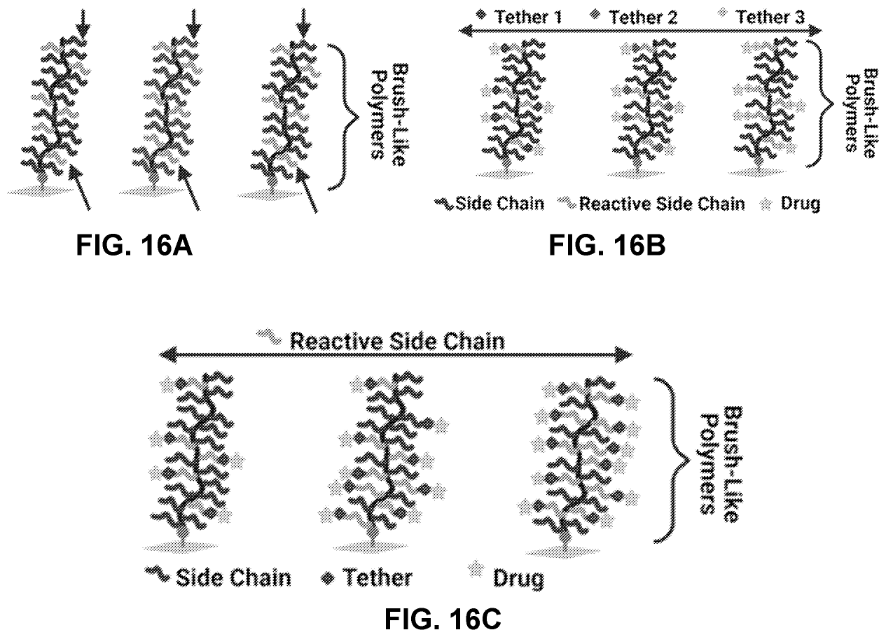

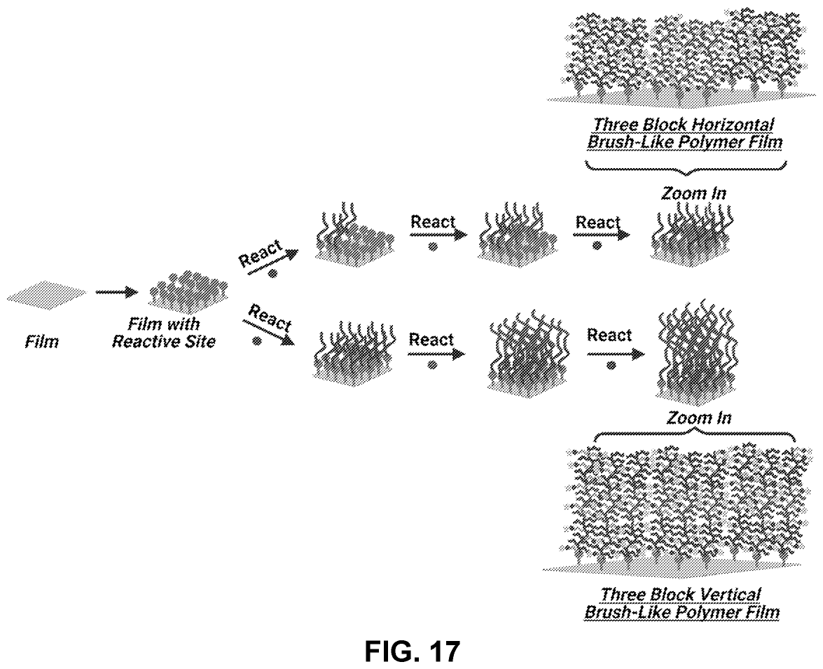

Methods for preparing silk fibroin surfaces with polymer chains may be classified as “grafting to” or “grafting from,” where the former couples a polymer to the surface and the latter grows polymer from the surface (FIGS. 14A-14B). “Grafting to” has been used to functionalize silk surfaces with polyethylene glycol (PEG) using cyanuric chloride-functionalized PEG (targets tyrosine residues) to reduce contact angle with water, with alkyne functionalized PEGs (targets tyrosine, histidine, and lysine residues) to reduce attachment of human stem cells and platelets, and with heparin (targets radicals generated on plasma treated surfaces) to reduce blood coagulation and increase cell proliferation on scaffolds. “Grafting to” has also been used to functionalize silk solutions with poly(amino acids), for the formation of hydrogels and layer by layer assemblies, and with glycopeptides, for binding of lectins. A recurring challenge with these approaches is that the theoretical number of residues that may be modified is small, therefore there may be a low likelihood that a reactive end of a polymer meets and reacts with a site on the surface. Accordingly, in various instances, monomers were employed, which have higher diffusion coefficients than polymers, to react with the silk surface and grow brush-like polymers using the “grafting from” method.

Silk fibers and fabrics have been modified by “grafting from” using free radical polymerization, redox polymerizations, and controlled radical polymerizations for textile applications that afford control over wettability or increase flame retardancy and antibacterial properties. Silk biomaterial surfaces have been modified using “grafting from” and free radical polymerization to grow acrylate polymers with 2-methacryloyloxyethyl phosphorylcholine side chains, where the resulting surfaces reduced platelet attachment compared to control silk surfaces, and with ferulic acid side chains, where the resulting surfaces increased the whole blood clotting time compared to untreated silk surfaces. Plasma-modified silk surfaces have also been used to grow polymers of poly(2-hydroxymethacrylate) (PHEMA) and poly(acrylic acid) (PAA) by free radical polymerization, where the PAA brushes were then subsequently modified with poly(ethylene glycol) (PEG, 750 Da) to alter surface hydrophilicity.

In some instances, though the PAA surface was more hydrophilic than PHEMA, HeLa cells were found to attach in larger amounts to PAA compared to PHEMA and unmodified silk. Without being bound to any theory, this finding may be because higher amounts of protein were adsorbing to the PAA surface. Conjugating PEG to PAA reduced cell attachment to the level of the unmodified silk, but not below.

Zwitterionic polymers may reduce membrane biofouling. In addition, zwitterions may be used to reduce platelet adhesion, prolong blood clotting, and lower the attachment of proteins to hydrogels to reduce the foreign body response. Accordingly, in some instances, zwitterionic moieties were incorporated on silk materials. (Jiang et al. J. Wuhan Univ. Technol. Mater. Sci. Ed. 2010, 25 (6), 969-74).

Accordingly, disclosed herein is a new synthetic route to generate brush-like polymers on silk protein film surfaces. Further disclosed herein is an example of an approach to grow brush-like polymers from the surface of degradable silk films, where the films were enriched with hydroxyl groups, functionalized with an initiator (e.g., bromoisobutyryl bromide (BIBB)), and finally reacted with acrylate monomers using atom transfer radical polymerization. Two different routes to hydroxyl enrichment were investigated, one involving reaction with ethylene oxide (EO) and the other using a two-step photo-catalyzed oxidation reaction.

More specifically, fibroin films were modified using ethylene oxide or a two-step photo-catalyzed oxidation method to overcome silk's limited number of reactive sites, two approaches that are expected to be translatable to other proteins. Brush-like polymers were then synthesized using surface-initiated atom transfer radical polymerization (ATRP) with two different monomers, one containing a zwitterionic pendant group and the other containing a PEG pendant group. The zwitterionic monomer was selected because its permanent dipoles may promote interactions between neighboring chains, and because it is commercially available with a chemical composition that may be less susceptible to hydrolysis than zwitterionic polymers that employ phosphoester groups.

The presently disclosed methods are synthetically distinct from previous reports of silk films functionalized with polymer chains, which have focused on attaching preformed polymers to the protein (“grafting to,” FIG. 14A) or polymerizing from (“grafting from,” FIG. 14B) the surface of unmodified or plasma treated silk materials that have a low number of reactive sites. The presently disclosed methods provide a greater amount of polymer conjugation as well as the appearance of a more uniform surface conjugation; this, in turn, translates into larger reductions of protein and cell attachment to the silk surfaces. Further, these experiments reveal the importance of protein secondary structure (altered via film processing) on the success of the polymerization, which is expected to provide an additional way to tune the reactivity of silk materials in synthetic reactions.

### IV. Exemplary Applications

Different iterations of the disclosed brush-like polymers on silk surfaces may be adapted for use in various applications, and/or in forming novel compositions, and/or articles. For example, the disclosed modified silk materials have lower cell and protein attachments and therefore are useful when enhanced diffusion around surfaces is required, such as, for example, in bioresorbable implants, wound dressing/tissue sealants, sutures, and drug delivery implants.

### A. Bioresorbable Implants

In various instances, the disclosed brush-like polymers on silk surfaces may be used to form bioresorbable implants, such as bioresorbable silk tubes, e.g., for blood vessel repair/replacement, and/or bioresorbable silk scaffold such as a tissue scaffold or wound dressing. As used herein, “bioresorbable” means a material is able to be resorbed or remodeled in vivo. The resorption process involves degradation and elimination of the original implant material through the action of body fluids, enzymes, or cells. The resorbed materials may be used by the host in the formation of new tissue, or it may be otherwise re-utilized by the host, or it may be excreted. The bioresorbable silk fibroin article described herein may have a resorption half-life ranging from a few hours to weeks to months. In various instances, the resorption half-life of the bioresorbable silk fibroin article described herein may be in a range of about 6 hours to about 4 weeks, about 12 hours to about 3 weeks, about 24 hours to about 2 weeks. In various instances, the resorption half-life of the bioresorbable silk fibroin article described herein may be at least about 1 months, at least about 2 months, at least about 3 months, at least about 4 months, at least about 5 months, at least about 6 months, at least about 7 months, at least about 8 months, at least about 9 months, at least about 10 months, at least about 11 months, at least about 12 months or longer. In various instances, the resorption half-life of the bioresorbable silk fibroin article described herein may be about 1 month to about 3 months, or about 3 months to about 6 months, about 6 months to about 12 months, or about 12 months to about 24 months.

### B. Wound Dressing/Tissue Sealants

In various instances, the disclosed brush-like polymers on silk surfaces may be used in a solution for applications where rapid gelation is needed, e.g., for treatment of a wound, e.g., to stop bleeding.

### C. Sutures

In various instances, the disclosed brush-like polymers on silk surfaces may be used to produce silk fibers with enhanced mechanical properties. Silk fibers have a variety of applications including, but not limited to, sutures and tissue engineering.

### D. Drug Delivery Implants

In various instances, a drug delivery device (e.g., an implantable microchip or scaffold, or an injectable drug depot) or wound dressing (e.g., a bandage or an adhesive) may comprise the disclosed brush-like polymers on silk surfaces and at least one active agent. In various instances, the active agent is covalently linked to the brush-like polymer at one of the polymer side chains.

### V. Experimental Examples

Without limiting the scope of the instant disclosure, various experimental examples of embodiments discussed above were prepared and the results are discussed below.

### A. Materials

Bombyx mori silkworms were obtained from Tajima Shoji Co., Ltd. (Tokyo, Japan). Lithium bromide (LiBr, ≥99%), chloroform (anhydrous, ≥99%), α-bromoisobutyryl bromide (BIBB, 98%), copper (I) bromide (CuBr, 99.999%), triethylamine (Et₃N, ≥99%), 2-(methacryloyloxy)ethyl]dimethyl-(3-sulfopropyl) ammonium hydroxide (DMAPS, 95%), poly(ethylene glycol) methacrylate (PEGMA, Mₙ 360), 2,2′-Bipyridyl (bipy, ≥99%), sodium carbonate (Na₂CO₃, ≥99%), sodium bicarbonate (NaHCO₃, BioReagent), ammonium persulfate (APS, ≥98%), sodium borate (BioXtra, ≥99.5%) and protease from Streptomyces griseus (type XIV) were purchased from Sigma Aldrich (St. Louis, MO). Ethylene oxide (99.9%) was purchased from Praxair (Slaterville, RI). Methanol (ACS certified), Triton X-100 (electrophoresis grade), acetone (ACS certified), regenerated cellulose dialysis tubing (3500 MWCO), Dulbecco's Modified Eagle Medium (DMEM), fetal bovine serum (FBS, qualified), antibiotic-antimycotic (Anti-Anti), CBQCA Protein Quantitation Kit, and phosphate buffered saline (PBS), were obtained from Fisher Scientific (Waltham, MA). CellTiter 96® AQueous One Solution Cell Proliferation Assay (MTS) was purchased from Promega (Madison, Wisconsin). Human mesenchymal stem cells (hMSCs) were obtained from Lonza (Walkersville, MD). Methoxypoly(ethylene glycol) activated with cyanuric chloride (mPEG-CC) was purchased from Creative PEGWorks (Chapel Hill, NC). Ultrapure water (dH₂O, 18 MOhm cm) was obtained from an in-house purification unit.

### B. Experimental Methods

i. Extraction of Silk Fibroin and Film Casting

Silk fibroin (SF) was extracted using a previously established protocol (Rockwood et al. Nat. Protoc. 2011, 6, 1612-31, SEQ. ID No. 1). Briefly, 5 g of cut cocoons were boiled in 2 L of 0.02 M Na₂CO₃ for 5 min. The fibers were rinsed with deionized water (dH₂O) three times for 20 min and dried overnight. After drying, the fibers were dissolved in 9.3 M LiBr (5 mL LiBr per gram dry fiber) for 4 hours at 60° C. The resulting solution was dialyzed (3500 MWCO) against dH₂O for 48 h, where the water was exchanged 6 times. The impurities of the purified solution were removed by centrifuging at 12700 g for 20 min at 6° C. The typical concentration of the solution obtained was 5 w/v %. For all of the following experiments, the solution was cast into films. The films were fabricated by adjusting the solution concentration to 1 w/v % and applying 500 μL to a polystyrene well (Corning Costar, 1.9 cm² area) and allowing to dry overnight, resulting in an “As Cast” SF film (denoted here as “SF As Cast”).

ii. Enrichment of Hydroxyl Groups on Silk Surfaces

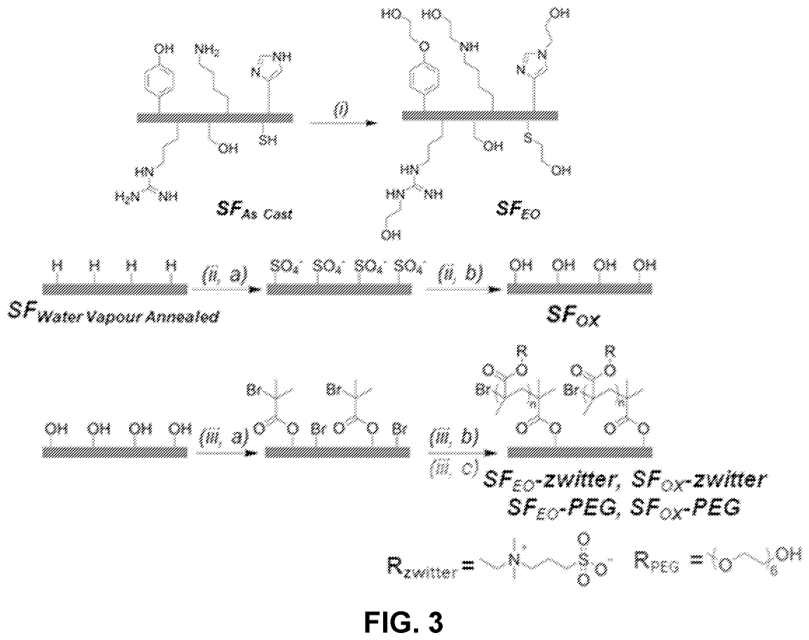

To increase the number of reactive groups for the polymerization, the surface of the SF films was modified to increase hydroxyl content through two different routes, one involving ethylene oxide coupling and the other involving a surface oxidation reaction. A flow chart overview of the synthesis steps is shown in FIG. 3.

iii. Ethylene Oxide Modification

“As Cast” SF films (SF_{As Cast}) were added to a vacuum reactor before applying an airtight seal. The reactor was degassed under vacuum and vacuum was held for 30 min until a pressure of 10-9 to 10-10 mbar was obtained. Ethylene oxide (3.2 mL, 64 mmol), which was condensed and dried over calcium hydroxide, was added to the reactor. Then, the reactor was removed from the vacuum line and placed in a 60° C. oil bath, where it was allowed to react for 72 h. The SF films, referred to as “SF_{EO}” were removed, rinsed with acetone to remove residual ethylene oxide, and dried at 60° C. for 48 h.

iv. Surface Oxidation Reaction

The photo-catalyzed surface oxidation reaction was motivated by a report that modified the surfaces of synthetic polymers (Yang et al. Polymer (Guildf). 2003, 44 (23), 7157-64). “As Cast” SF films (SF_{As Cast}) were rendered water-insoluble prior to oxidation via water vapor annealing. To water vapor anneal, silk films were dried overnight and transferred to a desiccator that contained liquid water in the bottom of the chamber. The films were suspended above the liquid, and vacuum was generated in the chamber using a water aspirator. After 10 minutes, the chamber was sealed to vacuum, and the films were annealed for 24 hours at 25° C., where the resulting films are denoted “SF_{WVA}”. Next, 200 μL of 15 w/v % ammonium persulfate (APS) in dH₂O was applied between two SF_{WVA} films and this layered film assembly was exposed to ultraviolet light (254 nm) for 5 min. This confined film assembly was used to promote the generation of SO₄²⁻ groups that are hydrolyzed to form hydroxyl groups while minimizing the formation of carboxylic acid groups. After UV exposure, the films were rinsed with dH₂O to remove excess APS and subsequently incubated in dH₂O for 30 min at 25° C. The films were then rinsed with acetone for 5 min and water for 5 min, alternating for a total of 3 times in each solvent, before drying at 60° C. for 48 h. These modified films are referred to as “SF_{OX}”.

### V. Initiator Attachment

The modified SF films (SF_{EO} or SF_{OX}, typical size 1.9 cm²) were thoroughly dried for 72 hours at 80° C. before adding to a round bottom flask that contained anhydrous chloroform (100 mL) and a stir bar. Then, triethylamine (1 mL, 7 mmol) was added to the mixture before purging the vessel with argon for 30 min. α-Bromoisobutyryl bromide (BIBB, 0.25 mL, 2 mmol) was added dropwise to the reaction and the reaction was allowed to proceed for 4 hours at 40° C. The films were thoroughly rinsed with acetone to remove any unreacted reagents and were dried overnight at 60° C. These films are referred to as “SF_{EO}-Br” if they originated from the ethylene oxide functionalized films and SF_{OX}-Br if they originated from the surface oxidized films.

vi. Atom Transfer Radical Polymerization

Acrylate monomers with two different types of pendant groups were selected to investigate the effect of the brush-like polymer's composition on the surface properties of the films. One monomer contains a polyethylene glycol (PEG) pendant group, where PEG is an uncharged polymer frequently employed to increase hydrophilicity and reduce biofouling. The other monomer contains a zwitterionic pendant group, where the hydrophilic zwitterionic group is charged, yet net neutral.

### vii. “Grafting From” Polymerization of PEGMA

Copper (I) bromide (CuBr, 15 mg, 0.1 mmol), 2,2′-Bipyridyl (Bipy, 31.2 mg, 0.2 mmol), and initiator attached films (SF_{EO}-Br or SF_{OX}-Br) were added to a Schlenk tube equipped with a stir bar, and the vessel was purged with argon for 30 min. Poly(ethylene glycol) methacrylate (PEGMA, 0.489 mL, 1.5 mmol) was dissolved in dH₂O/methanol (1:4 v:v) and the solution was purged with argon for 30 min before transferring to the Schlenk tube. The polymerization proceeded for 12 hours at 25° C. The films, denoted as “SF_{EO}-PEG” or “SF_{OX}-PEG” where the subscript again denotes the hydroxyl enrichment method, were rinsed with methanol, and sonicated in dH₂O to remove any bound or unreacted reagents. The films were dried at 60° C. and stored in a desiccator.

viii. “Grafting From” Polymerization of DMAPS

Copper (I) bromide (15 mg, 0.1 mmol), Bipy (31.2 mg, 0.2 mmol), and SF-Br films were added to a Schenk tube, which was then purged with argon for 30 min. [2-(Methacryloyloxy)ethyl]dimethyl-(3-sulfopropyl) ammonium hydroxide (DMAPS, 0.419 g, 1.5 mmol) was added to a solution of dH₂O/methanol (1:1 v:v) and was purged with argon for 30 min. The monomer solution was added to the Schlenk tube, and the reaction proceeded for 12 hours at 25° C. After polymerization, the films were rinsed with methanol, sonicated in deuterated water (dH₂O), and dried at 60° C. before storing in a desiccator. The resultant films are denoted as “SF_{EO}-zwitter” or “SF_{OX}-zwitter” where the subscript denotes the hydroxyl enrichment method.

ix. PEG Attachment to Silk Films by “Grafting To”

Silk films were cast into wells with an area of 9.6 cm² from a 1% w/v solution and allowed to dry overnight. The films were rendered insoluble by water vapor annealing for 24 hours at 25° C. The PEGylation reaction proceeded as previously reported by Vepari et al. (J. Biomed. Mater. Res.—Part A 2010, 93 (2), 595-606). Briefly, the films were incubated in a solution of 0.02 M sodium borate buffer (pH 9) for 30 min at 25° C. A solution of methoxypoly(ethylene glycol) activated with cyanuric chloride (mPEG-CC) was prepared at a concentration of 62.5 mg/mL in 0.02 M sodium borate (pH 9), and 0.998 mL of the solution was added to each silk film. The films were allowed to react with the solution at 4° C. overnight. The product, denoted as “SF-PEG_{CG},” were rinsed three times with sodium borate buffer to remove unreacted mPEG-CC.

x. Attachment of CPADB on the Silk Fibroin.

Dried SF_{OX} films were added to a round bottom flask containing 4-cyano-4-(phenylcarbonothioylthio)pentanoic acid (CPADB, 0.1 g, 0.36 mmol) dissolved in 10 mL of anhydrous dichloromethane (DCM). Then, N,N′-dicyclohexylcarbodiimide (DCC, 0.1 g, 0.48 mmol) was dissolved in 5 mL dichloromethane and added to the reaction mixture before purging the vessel with argon for 30 min. 4-Dimethylaminopyridine (DMAP, 0.055 g, 0.45 mmol) was dissolved in 10 mL DCM and slowly added to the reaction mixture and the reaction was allowed to proceed for 72 h at 25° C. The films were thoroughly rinsed with DCM to remove any unreacted reagents and dried overnight at 60° C. The resulting films are termed as “SF-CPADB.”

xi. Reversible Addition Fragmentation Chain Transfer (RAFT) Polymerization.

Methacrylate monomers with varying hydrophilicities were selected to generate brush-like polymers on the surface of SF films. MMA, HEMA, AEMA and DMAPS were employed for graft polymerization.

xii. “Grafting from” Polymerization of Methacrylate Monomers.

The dried SF-CPADB films and 2,2-azobis(2-methylpropionitrile (AIBN) were added to a Schlenk tube, which was then purged with argon for 30 min. The molar ratio of CPADB to AIBN was 4:1. The monomer (MMA/HEMA/DMAPS/AEMA) was dissolved in dimethyl sulfoxide (DMSO) at 500 mM concentration and was purged with argon for 30 min. Then the solution was added to the Schlenk tube, and the reaction proceeded for 24 h at 70° C. in an oil bath. After polymerization, the films were rinsed with DMSO and dried at 60° C. under vacuum before storing in a desiccator. The grafted films (single blocks) are denoted as “SF-Monomer” based on the type of monomer used for the polymerization.

xiii. Random Copolymerization of Methacrylate monomers with AEMA.

Methacrylate monomers (MMA, HEMA, and DMAPS) were each randomly copolymerized with AEMA. In a typical procedure, dried SF-CPADB and azobisisobutyronitrile (AIBN) were placed in a Schlenk tube while purging with argon for 30 min. A molar ratio of 4:1 was used for 4-cyano-4-(phenylcarbonothioylthio) pentanoic acid (CPADB) to AIBN. The monomer (MMA or HEMA or DMAPS) and AEMA were dissolved in DMSO at 1000 mM concentration respectively. Then both solutions were added to the Schlenk tube, allowing the reaction to continue for 24 h at 70° C. in an oil bath. Following the reaction, the films were soaked and rinsed with DMSO to remove any loosely attached free polymer from the solution and dried at 60° C. under reduced pressure. The modified films (random blocks) are referred to as “SF-Monomer+AEMA”.

### C. Characterization Methods

i. Attenuated Total Reflectance-Fourier Transform Infrared (ATR-FTIR) Spectroscopy

Attenuated total reflectance-Fourier transform infrared (ATR-FTIR) spectroscopy (Magna 560 FTIR spectrometer (Madison, WI) with a diamond ATR accessory (Specac, Fort Washington, PA)) was used to observe changes in functional groups after synthesis and processing steps. After each modification reaction and subsequent drying, the spectra of films were collected by scanning from 4000-400 cm⁻¹ (step size 4 cm⁻¹). ATR-FTIR was also used to quantify beta sheets in the films by deconvoluting the Amide I region using Origin software (v. 8.1, Northampton, MA) and established methods (Hu et al. Macromolecules 2006, 39, 6161-70).

ii. Contact Angle Measurements

To quantify the changes in surface hydrophilicity, water contact angle was measured using a sessile drop method (Ramé-Hart goniometer, Mountain Lakes, NJ). After each modification step, the contact angle was measured by depositing a 5 μL drop of dH₂O. Because the SF-zwitter films exhibited a very low contact angle from the sessile drop method, the captive bubble method was also used to quantify the contact angle (Pendant Drop Tensiometer, Westbury, NY) of films. The captive bubble measurement was carried out by affixing the films onto a glass slide and placing the slide over a beaker containing dH₂O. Next, an angled needle was used to apply an air bubble with an approximate volume of 20 μL to the film, and the angle between the sample and air bubble was measured.

iii. Atomic Force Microscope (AFM) Visualization

Transformations of the silk surface morphology were visualized using an atomic force microscope (AFM) (Asylum Research MFP-3D, Goleta, CA). Samples were dried and mounted onto glass slides with double-sided tape and pressed down using a freshly cleaved mica surface to ensure complete physical contact between the glass slide and the sample. Imaging was performed in tapping (also known as AC) mode using silicon tips (Asylum Probes, AC-160). Scans sizes of 5×5 μm were acquired at line rates of 1 Hz, with typical set point and feedback gain settings optimized for surface tracking.

iv. Degradation of Films

Unmodified SF films are degradable by hydrolysis or using enzymatic treatments at a rate that has been shown to depend on beta sheet content, but it was questioned how the addition of brush-like polymers would affect the degradation rate. Films were prepared as described above and dried at 60° C. before beginning the study. The films were then added to pre-weighed microcentrifuge tubes and 1 mL of 1×PBS (control) or 1×PBS containing 1 U/mL protease XIV was added to the tube. Every 2 days, the tubes were centrifuged (10,000 g, 10 min, 25° C.) and the solution was decanted. The films were rinsed with deionized water (dH₂O) and centrifuged again. The water was again decanted, and the tubes were allowed to dry for 1 hours at 60° C. before measuring the mass. The percent mass remaining, m, at each time point was determined by formula (1):

$$m = \frac{{m}_{t}}{{m}_{o}} \cdot 100 \qquad ( 1 )$$

where mₜ is film's mass at the time measured in the study and mₒ is the film's initial mass.
v. Adsorption of Bovine Serum Albumin.

A protein adhesion study was performed to quantify how the synthesis method and chemical composition of the brush-like polymers affect protein attachment to SF films. Larger films were required for this study because the limit of detection of the CBQCA (3-(4-carboxybenzoyl) quinoline-2-carboxaldehyde) assay. The films were cast by applying 2.5 mL of 1% w/v SF solution to the wells of 6 well plates (9.6 cm² per well) and drying at room temperature. The subsequent reactions were carried out as described above to generate the brush-like polymer surfaces. Films were then immersed in 10 mg/mL bovine serum albumin (BSA) overnight at 6° C. After incubation, the films were gently washed with 750 μL dH₂O six times to remove loosely attached protein. The films were then incubated with 500 μL of 0.1% v/v Triton X-100 for 1 hours at 25° C. to detach the adsorbed BSA. The solutions were collected and assayed with using a CBQCA working solution (2 mM CBQCA in 0.1 M sodium borate). The fluorescence (Ex 465 nm, Em 550 nm) was measured, and BSA was calculated based on a standard curve.

vi. Attachment of Human Mesenchymal Stem Cells (hMSCs)

The attachment of hMSCs to the silk films was measured using an MTS assay, which detects viable cells using a tetrazolium compound that is reduced into a formazan dye and detected spectroscopically. The films used in this experiment had an area of 0.95 cm² but otherwise followed the same synthetic routes described above. Films were sterilized before use by soaking in 70% v/v ethanol for 24 h, rinsing six times with sterile dH₂O, and placing in a sterile 48 well plate. hMSCs (Passage 4) were seeded on the films at a high seeding density (105,000 cells/cm²). At each time point (1 h, 3 h, 6 h, and 24 h), the films were gently rinsed with 1×PBS to remove any loosely attached cells and transferred to a new sterile 48 well plate. Then, 240 μL of complete medium (89% DMEM, 10% FBS, and 1% anti-anti) and 20 μL of CellTiter 96® AQueous One Solution Reagent (MTS) was added to each well. The samples were incubated for 1 hours at 37° C. Next, 100 μL of the medium was transferred to a 96 well plate, and absorbance at 490 nm was measured with a UV plate reader (BioTek, Winooski, VT). The number of cells attached to films was calculated using a standard curve generated from cells seeded on tissue culture plastic at varying densities.

### D. Summary/Results

i. Synthesis of Polymer Brushes on Silk Fibroin Films.

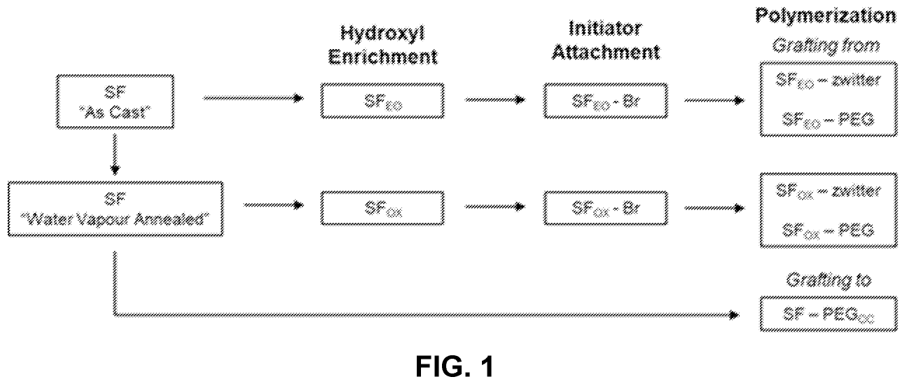

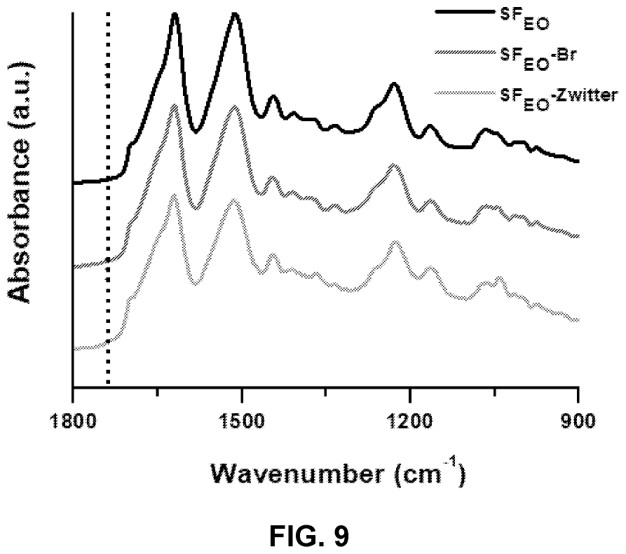

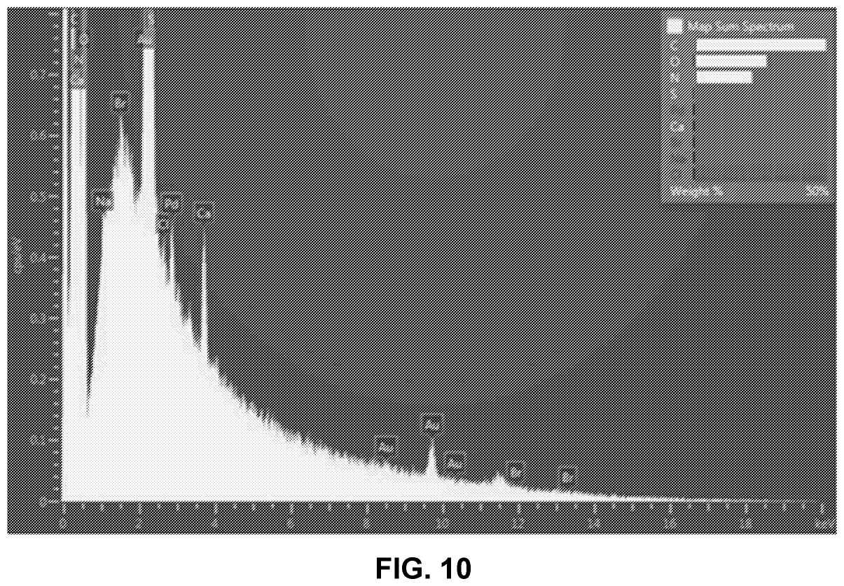

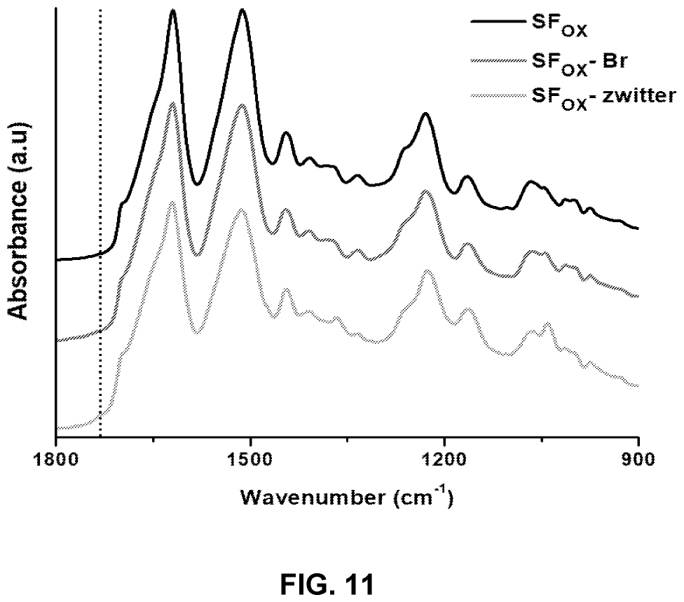

Preliminary experiments polymerized the zwitterionic monomer (DMAPS) from the surface of unmodified silk films using atom transfer radical polymerization (ATRP). These films were methanol annealed, meaning “As Cast” films (SF_{As Cast}) were soaked in a 95% methanol/5% water solution for 24 hours at 25° C. to induce beta sheets and render the films insoluble in water before reacting with initiator and polymerizing with DMAPS. The ATR-FTIR spectra of the resulting films showed very low levels of the sulfonate group, indicating that the number of brush-like polymers on the surface of the film and/or the molecular weight of chains was low (FIG. 9). Without being bound by theory, it was hypothesized that this result may be because the hydroxyl groups in silk fibroin are mostly found in serine residues, which are frequently located in the beta sheet regions of the protein. This observation motivated the enrichment of the hydroxyl content on the film's surface and to use methods that keep beta sheet content in the films low. To keep beta sheet content low, water vapor annealing was used to induce beta sheets instead of methanol annealing, and water vapor annealing was only employed to render the film insoluble for the oxidation reaction because this reaction uses water as a solvent (FIG. 1).

To enrich hydroxyl content, two methods were investigated to modify the surface of SF films. The first method (FIG. 3, i) reacted the films with ethylene oxide (EO), which targeted the tyrosine, lysine, serine, cysteine, histidine, and arginine. This product, denoted as “SF_{EO},” has a theoretical degree of hydroxylation of about 18.2 mol %. The second method (FIG. 3, ii, a-b) used a surface oxidation method previously reported in literature. The oxidation mechanism is proposed to proceed by abstracting a hydrogen atom from the film's surface to form tertiary SO₄⁻ groups, which are susceptible to hydrolysis in aqueous media, resulting in the generation of hydroxyl groups. The potential for hydrogen abstraction is dependent on the bond strength, meaning that hydrogens attached to tertiary carbons are more susceptible to abstraction than hydrogens attached to primary carbons. Since this reaction targets surface hydrogen atoms, it has the potential to modify the hydrogen of the amide backbone in addition to the hydrogens attached to tertiary or secondary carbons in the side chains of valine, leucine, isoleucine, proline, methionine, lysine, arginine, glutamine, and glutamic acid residues. In total, these amino acids amount to 5.6 mol % of a silk molecule but given the reactivity at the hydrogen of the amide backbone, it is difficult to determine a precise theoretical degree of modification of the product, SF_{OX}. Because the number of potential reaction sites for the oxidation reaction, it should be noted that FIG. 3 ii, a-b does not exhibit all possible modifications.

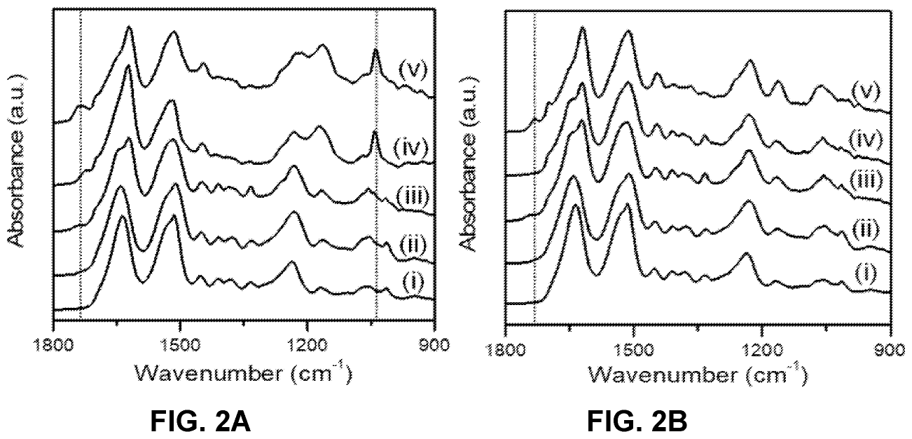

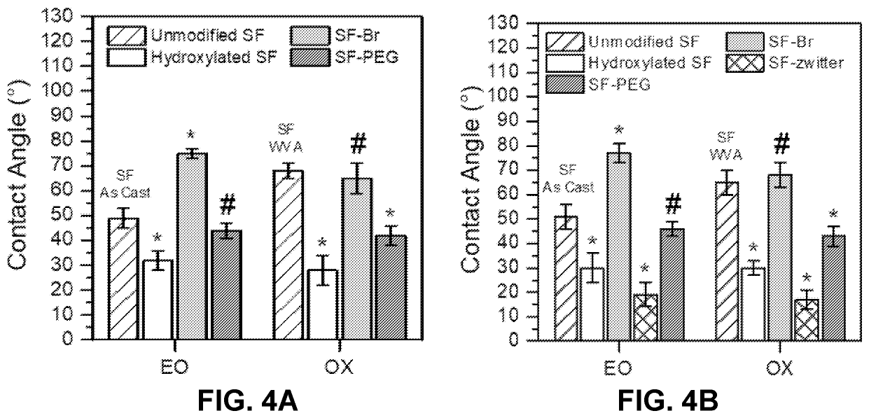

To confirm successful hydroxyl substitution, the surfaces of the unmodified and modified silk films were characterized using ATR-FTIR (FIGS. 2A-2B) to assess functional groups and by measuring contact angle with water to characterize surface hydrophilicity (FIGS. 4A-4B). The ATR-FTIR spectrum for SF_{EO} (FIG. 2A, trace ii; FIG. 2B, trace ii) did not result in the generation of new peaks compared to the unmodified silk surface (FIG. 2A, trace i; FIG. 2B, trace i), but the peak from the hydroxyl substitution is expected to overlap with the hydroxyl of the native protein, therefore this result was expected. The ATR-FTIR spectrum for SF_{OX} (FIG. 2A, trace iii; FIG. 2B, trace iii) showed the presence of a small peak at ˜1740 cm⁻¹ due to the formation of carboxyl groups, indicating that some formed despite this being discouraged by the experimental setup, and the signal from the hydroxyl groups was difficult to resolve again due to hydroxyl of the silk film.

To assist in confirming surface modification by EO and by OX methods, the surface hydrophilicity was characterized by contact angle measured using two different methods: sessile drop (FIG. 4A) and captive bubble (FIG. 4B). The unmodified SFs used in the EO and OX syntheses had different processing methods, where the SF film used in the EO synthesis was used “As Cast” (SF As Cast) and the SF film used in the OX synthesis was water vapor annealed (SF_{WVA}) (FIG. 1), which contributes to their different values and is consistent with literature. Compared to the unmodified SF, the water contact angle of the hydroxylated films, SF_{EO} and SF_{OX}, decreased (FIGS. 4A-4B), suggesting that the reaction was successful at increasing hydrophilicity. After modification with EO, the contact angle decreased to about 30°, from about 50° for SF_{As Cast} (FIG. 4A). This was expected due to the increase in OH groups on the surface, making the film more hydrophilic. Film modification by oxidation also shows a decrease in contact angle to about 30°, which is a significant change from the unmodified SF (SF_{WVA}), which was approximately 65° (FIG. 3A). SF_{EO} and SF_{OX} have similar contact angles, indicating that their surface hydrophilicity is similar.

The modified SFs (SF_{EO} and SF_{OX}) were then reacted with α-bromoisobutyryl bromide (BIBB) to terminate the surface with an initiator suitable for subsequent reactions using atom transfer radical polymerization (ATRP). To assess successful initiator attachment, ATR-FITR and scanning electron microscopy (SEM, FEI Nova NanoSEM 450, Hillsboro, OR) with energy-dispersive X-ray analysis (EDS, Oxford AZtecEnergy Microanalysis System with X-Max 80 Silicon Drift Detector, Concord, MA) (SEM-EDS) were used, but it was found that the C—Br signal in the FTIR spectrum (FIG. 9) was obscured by the silk protein signal and SEM-EDS signal was also small compared to the noise (FIG. 10). In lieu of direct assay for bromine presence, water contact angle was used to confirm the successful attachment of the initiator. After initiator attachment, the contact angles for SF-Br sharply increased from ˜30° to ˜75° for the EO synthesis and 65° for the OX synthesis (FIG. 4A). The change in surface hydrophilicity is consistent with the attachment of hydrophobic initiator.

Using the initiator-attached films, SF-Br, polymers were “grafted from” the surface of silk by surface-initiated ATRP (SI-ATRP). Two acrylate monomers were selected for investigation. The first contained a polyethylene glycol (PEG) pendant group that is hydrophilic and lacking in charged groups. The second monomer was hydrophilic and zwitterionic, where the charged groups were balanced to obtain an electrically neutral monomer. ATR-FTIR and contact angle were used to confirm successful surface polymerization of the brush-like polymers. Herein, the term “brush-like” is used to describe the polymers because they are synthesized using the same chemistries that may produce polymer brushes, but a direct measure of surface density of the chains to prove chain interactions was not possible, thus the term “brush-like” was used. For both SF_{EO}-zwitter and SF_{OX}-zwitter, the ATR-FTIR spectra (FIG. 2A, traces iv and v) show a new peak due to the formation of the ester bond from the polymerization at ˜1720-1730 cm⁻¹, and a peak due to the sulfonate (—OSO₃⁻) group of the zwitterionic polymer is present at 1040 cm⁻¹. The amount of sulfonate may be quantified by normalizing the amide II region (1500-1600 cm⁻¹) of SF-zwitter spectra and determining the area of the sulfonate peak at 1040 cm⁻¹. The areas of SF_{EO}-zwitter and SF_{OX}-zwitter were 2.29 and 2.45 (relative to the normalized amide II region), respectively, suggesting that the oxidation method results in more brush-like polymer surface functionalization. This result may arise due to the greater number of reactive sites theoretically available to the oxidation reaction compared to the ethylene oxide reaction, though not all reactive sites will generate brush-like polymers because of other constraints, such as sterics. The ATR-FTIR spectra of SF_{OX}-PEG and SF_{EO}-PEG (FIG. 2B, traces iv and v) also show the appearance of a peak at 1730 cm⁻¹, indicating successful polymerization, however the PEG-containing brush-like polymers had no distinct new peaks, hence quantifying by peak deconvolution was not possible.

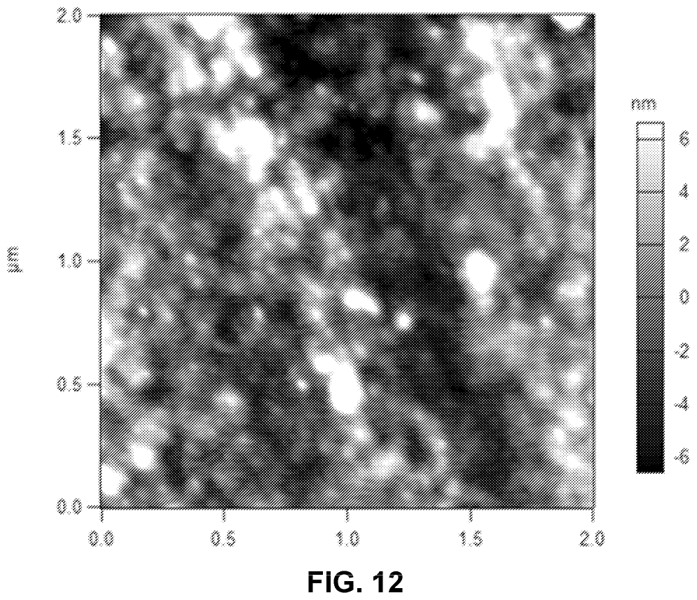

Because it was hypothesized that methanol annealing causes a transformation that reduces the accessibility of reaction sites on SF films, the photo-catalyzed oxidation reaction was run on films that were annealed by soaking in 95% methanol in water at 25° C. for 24 h. An initiator was then attached and ATRP polymerization of the zwitterionic monomer was attempted, but those films were found to display little brush-like polymer content (FIG. 12). This supports the proposed hypothesis that the annealing treatments of the silk films impacts their reactivity in these syntheses and validates the use of water vapor annealing instead of methanol treatment to render films insoluble prior to hydroxyl enrichment.

The contact angle of PEG-containing brush-like polymer surfaces could be measured using either the sessile drop or captive bubble methods. The zwitterionic-containing brush-like polymer surfaces were found to wet too rapidly to obtain an accurate measurement by sessile drop, therefore only the captive bubble method was used for those films. The contact angles of SF_{EO}-PEG and SF_{OX}-PEG are shown in FIG. 4A for the sessile drop and FIG. 4B for the captive bubble method, and the values are about 40°, which is a significant decrease from SF-Br and shows that the surface was also successfully modified with PEG-containing brush-like polymers. The contact angle for films with PEG-containing brush-like polymers was significantly lower than SF when the oxidation method (SF_{OX}-PEG) was employed, but this decrease was not found to be significant for the brush-like polymers prepared from the ethylene oxide modified surfaces (SF_{EO}-PEG). Using the captive bubble method, SF_{EO}-zwitter and SF_{OX}-zwitter (FIG. 4B) both show a contact angle of approximately 20°, a significant reduction from SF-Br that confirms successful functionalization. The captive bubble method (FIG. 4A) also shows that the SF_{EO}-zwitter and SF_{OX}-zwitter display significantly lower contact angles than SF_{EO}-PEG and SF_{OX}-PEG.

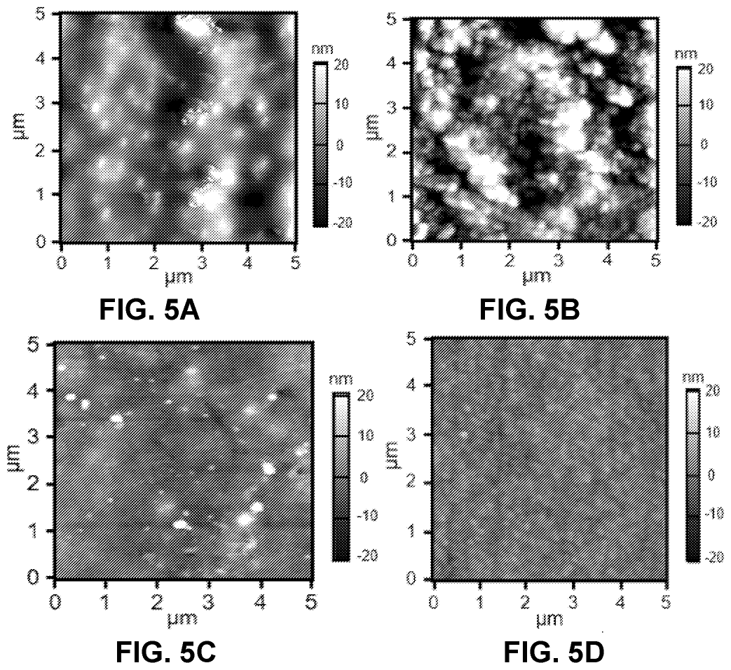

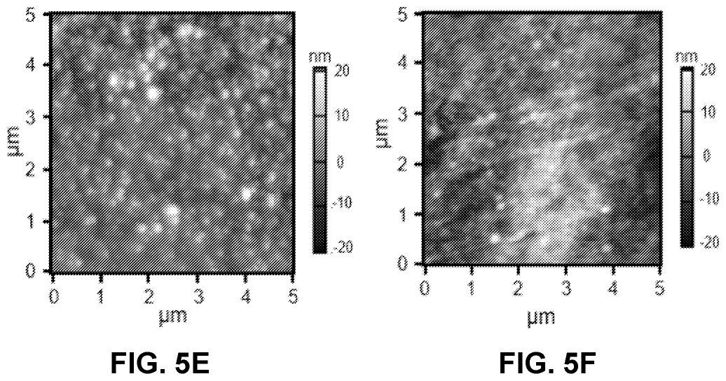

The changes in the surface morphology of the films were characterised by atomic force microscopy (AFM), and the height micrographs of the modified silks are shown in FIGS. 5A-5F. SF_{EO} (FIG. 5A) appeared to be more uneven than did the SF_{OX}, but both films modified with the brush-like polymers exhibited increased height when compared to SF_{EO} (FIG. 5E) and SF_{OX} (FIG. 5D), providing further evidence that the polymerisation was successful. Compared to the brush-like polymer films prepared on the surfaces modified with ethylene oxide, the brush-like surfaces prepared from the oxidized surfaces appeared to be more uniform. While both the ethylene oxide and the oxidation reactions led to brush-like polymers on the surface of the silk films, the next series of experiments were focused on the oxidized series of films due to their appearance of uniformity, greater brush-like polymer thickness based on FTIR measurements, and the potential for the future generation of patterned brush-like surfaces by employing masks during the light-catalyzed oxidation reaction.

ii. Beta Sheet Secondary Structure Formation

Silk fibroin is known to self-assemble into beta sheet secondary structures. Tuning beta sheet content affords the opportunity to tune SF's properties, including degradation and mechanical properties, therefore the changes in beta sheet content of the films as they progressed from “As Cast” films (SF As Cast) through the synthesis and processing steps to generate the brush-like surfaces (Table 1) were assessed. ATR-FTIR scans of all films were measured to analyze the amide I region (1600 to 1700 cm⁻¹), which is known to display spectral changes in response to changing protein structure. For example, peaks that correspond to random coils are positioned at 1650 cm⁻¹ and 1540 cm⁻¹ transition to 1625 cm⁻¹ and 1515 cm⁻¹, respectively (Hu et al. Macromolecules 2006, 39, 6161-70). The amide I region was deconvoluted into peaks corresponding to different secondary structures using established methods (Hu et al. Macromolecules 2006, 39, 6161-70). Table 1 shows the beta sheet content measured after each reaction, without the application of any other treatments to deliberately trigger the secondary structure, such as exposure to water vapor or methanol. For the EO series, SF As Cast exhibited the lowest beta sheet content of all the films (about 19.7%). The subsequent reactions, the EO treatment and the initiator attachment, do not induce further changes in beta sheet content. This is expected because these reactions do not utilize solvents or other conditions that have been shown to change the conformation of silk. After the DMAPS and PEGMA polymerization, the beta sheet content increases to approximately 34-35%, which is a result of the exposure of films to methanol during the reaction. In the OX series, the SF_{WVA} films contain more beta sheet content than the SF As Cast films used in the EO series, a finding that is consistent to that previously disclosed by Hu et al. (Biomacromolecules 2011, 12 (5), 1686-96). Similar to the EO series, the surface oxidation and initiator attachment reactions do not induce beta sheets, but the DMAPS and PEGMA polymerization increase the beta sheet content. Again, this was expected due to the films being exposed to methanol during the polymerization.

TABLE 1
Beta sheet content of modified SF films
through synthesis and modification.

Beta Sheet Content (%)

| | EO¹ | OX² |
|---|---|---|
| Unmodified SF | 19.7 ± 4.19 | 27.5 ± 1.22 |
| | (this film is SF_{As Cast}) | (this film is SF_{WVA}) |
| Hydroxylated SF | 21.3 ± 3.68 | 29.0 ± 2.83 |
| SF-Br | 24.1 ± 2.35 | 28.4 ± 3.46 |
| SF-zwitter | 35.6 ± 2.99 | 35.2 ± 3.04 |
| SF-PEG | 34.0 ± 3.01 | 34.5 ± 2.18 |

¹Initial modification by ethylene oxide;
²Initial modification by photo-catalyzed oxidation.

The impact of the brush-like polymer surface modifications on the protein's ability to form beta sheet structures also was quantified. To complete this study, the films in the EO and OX series were first prepared as described above. Each film was then annealed by soaking in 95% methanol in water for 24 hours at 25° C., drying, and then analyzing beta sheet content using ATR-FTIR with peak deconvolution. The goal was to determine if the surface functionalization affect the final amount of beta sheet that may form. “As Cast” SF films (SF As Cast) soaked in the methanol solution under these conditions were found to display 36.7±2.30% beta sheet content. Table 2 shows that the hydroxylated SFs (SF_{EO} and SF_{OX}) and the SF films with brush-like polymers (SF-zwitter and SF-PEG) all show beta sheet contents similar to the “As Cast” unmodified SF films, indicating that the surface functionalization does not affect the ability of the protein film to form beta sheets.

TABLE 2
Beta sheet content of modified silk films
after exposure to 95 v/v % methanol.

Beta Sheet Content (%)

| | EO¹ | OX² |
|---|---|---|
| Hydroxylated SF | 34.3 ± 2.74 | 37.1 ± 3.38 |
| SF-zwitter | 36.3 ± 4.44 | 37.7 ± 3.92 |
| SF-PEG | 35.9 ± 2.50 | 36.5 ± 4.09 |

¹Initial modification by ethylene oxide (EO);
²Initial modification by oxidation (OX).

iii. Film Degradation

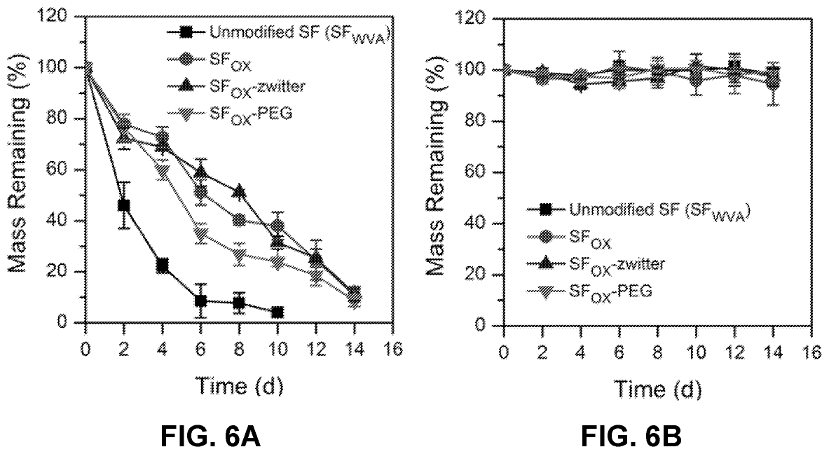

SF has been shown to degrade via hydrolysis or at an accelerated rate by treating with Protease XIV, the rate of which is dependent on its beta sheet content. It was not known how the degradation would be affected by the brush-like polymer surfaces. Films were exposed to 1 U/mL protease XIV in 1×PBS (enzyme) (FIG. 6A) or 1×PBS only (control) (FIG. 6B).

SF_{OX}, SF_{OX}-zwitter, and SF_{OX}-PEG films all show similar degradation profiles. This suggests that the presence of the brush-like polymers, though synthetic, do not hinder the ability of the film to degrade. SF was found to degrade faster and reach a lower mass than the modified films. The slower degradation of the functionalized silk surfaces is attributed to the lack of degradation of the polymer brushes and the decreased ability of the enzyme to attach to the more hydrophilic surfaces. The degradation profiles from incubation in 1×PBS are shown in FIG. 6B, where all compositions show resistance to degradation over the duration of the experiment (14 days). This result is consistent with the literature, where silk fibroin with beta sheet content degrades over much longer timescales without the presence of Protease XIV.

iv. Protein and Cellular Attachment Studies

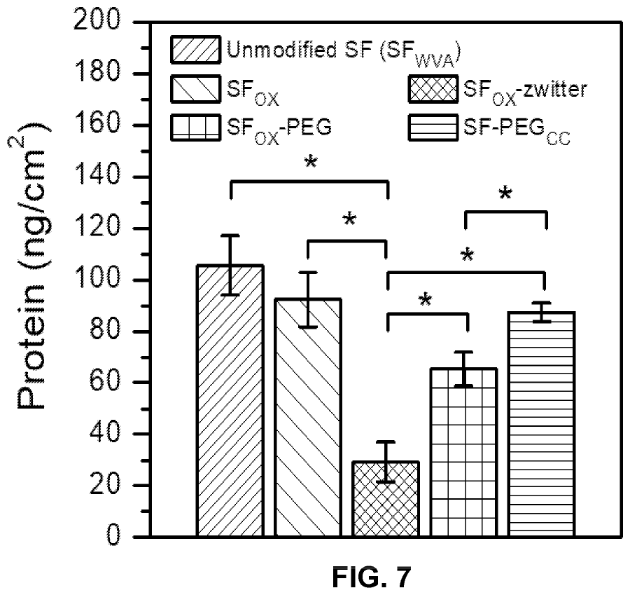

To evaluate the biofouling potential of the modified SF films, protein adsorption was quantified. Bovine serum albumin (BSA) was selected for investigation because it is a large component in blood, one of the first substances to deposit after a biomaterial is implanted and is the protein that is frequently used as a model foulant, including for applications in marine fouling and filtration membranes. The films were incubated with a solution of BSA, rinsed with water to remove loosely attached protein, and then treated with a surfactant solution. The surfactant was used to remove bound protein and was assayed to quantify the amount of protein adsorbed onto the surface, which was then divided by the area of the film (9.6 cm²) to obtain the values reported in FIG. 7. Films that were not exposed to BSA did not result in a detectable amount of protein in the surfactant solution, indicating that the signal from this assay is from the adsorbed BSA and not the release of silk protein from the film.

Compared to the water vapor annealed SF (SF_{WVA}), SF_{OX} showed a similar amount of BSA bound to the surface, despite the finding that SF_{OX} displayed a significantly lower contact angle (about 30° by captive bubble) than SF_{WVA} (about 65° by captive bubble). These results show that, though changes in contact angle may be observed, the addition of hydrophilic functional groups does not necessitate a reduction in protein attachment. The polymer chains “grafted from” the film's surface using ATRP (SF_{OX}-zwitter and SF_{OX}-PEG) showed a reduction in the amount of protein adhered to the surface. SF_{OX}-zwitter showed the lowest amount of BSA adhered to the surface, indicating the zwitterionic brush-like polymers display less fouling than the PEG-containing brush-like polymers.

To compare these results to the literature, a synthesis that conjugated a 5 kDa PEG molecule to silk fibroin surfaces using a “grafting to” method was replicated to result in the product SF-PEG_{CC}. The water contact angles of the surfaces are similar and not statistically different, specifically ˜42° for SF_{OX}-PEG and ˜45° for SF-PEG_{CC}. The molecular weights of the PEG containing chains are also similar: SF-PEG_{CC} chains are ˜5,000 g/mol and SF_{OX}-PEG chains are ˜8,000 g/mol (Table 3). The finding that SF_{OX}-PEG had less protein bound than SF-PEG_{CC} may be due to several factors.

TABLE 3
Molecular weight of polymers prepared using sacrificial initiator.

| Conjugate Reaction | Molecular Weight (g/mol) |
|---|---|
| Zwitter | 11,000 |
| PEGMA | 8,000 |

First, there may be a greater density of brush-like polymers on the SF_{OX}-PEG surfaces compared to SF-PEG_{CC} because of the use of “grafting from” to synthesize SF_{OX}-PEG. The chain architecture is also different (SF-PEG_{CC} has PEG in the backbone and is terminated with a methoxy group, but SF_{OX}-PEG has PEG pendant groups and is terminated with bromine) and may impact interactions between neighboring chains and the coverage of the film surface. While comparing the outcomes of protein attachment studies reported in literature is somewhat challenging due to differences in methods and how data are reported, the SF-zwitter surfaces have protein attachment of ˜29.2±7.7 ng/cm², which is lower than other methods to reduce attachment to SF surfaces. The surface density, molecular weight, and pendant group composition of the brush-like polymers may be varied depending on the desired attachment.

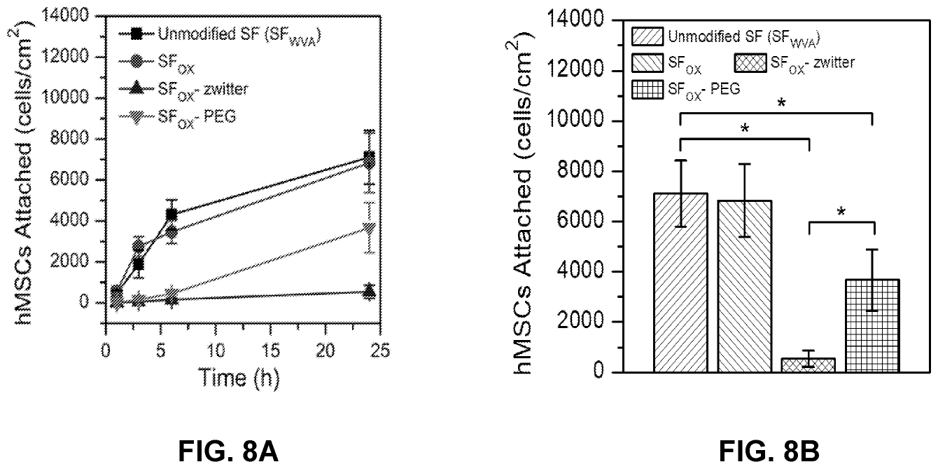

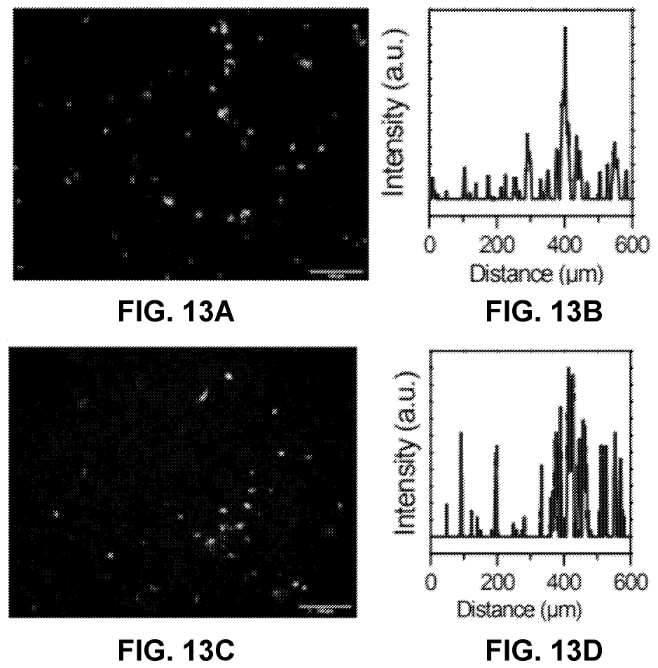

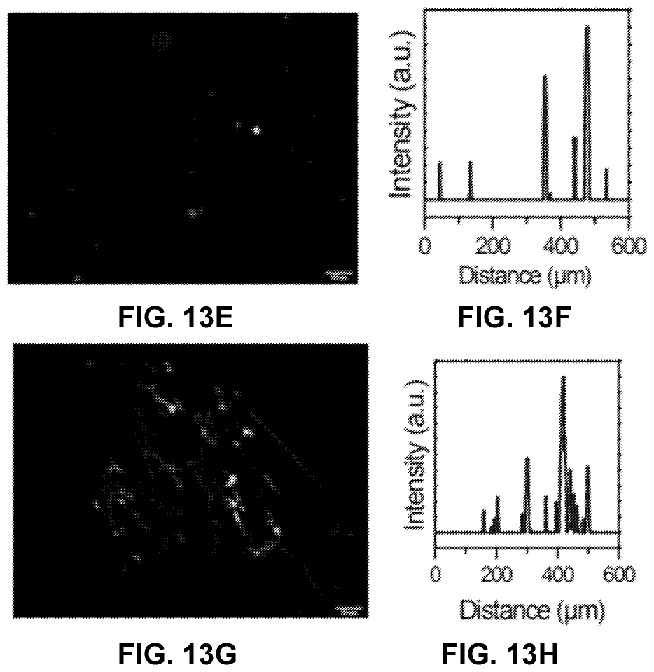

Cell attachment to the SF and modified SF films was investigated by seeding human mesenchymal stem cells (hMSCs) onto the films and quantifying the number of cells quantified after incubating for 1 h, 3 h, 6 h, and 24 h. Human mesenchymal stem cells (hMSCs) were selected for study due to their presence in the circulation and may migrate to wounded areas to start the process of tissue regeneration. The number of cells attached to the samples at each time point and after 24 hours are shown in FIG. 8A and FIG. 8B, respectively. A similar number of cells attached to the water vapor annealed silk (SF_{WVA}) and SF_{OX}, illustrating that even though SF_{OX} is more hydrophilic than SF, it is not necessarily more effective at repelling attachment of cells, a finding that is consistent with the protein adhesion experiments (FIG. 7). SF_{OX}-PEG depicted fewer cells adhered than SF_{WVA} or SF_{OX}, suggesting that the PEG brush-like polymers are effective at reducing fouling. SF_{OX}-zwitter exhibited the lowest number of cells attached, which is consistent with the protein adhesion results and highlights the zwitterionic brush-like polymer surface's resistance to biofouling. The data shown in FIGS. 8A-8B are consistent with images of cells seeded on the different films acquired at the 24-hour time point (FIGS. 13A-13H). Taken together, the protein and cell adhesion data indicate that both the PEG-containing brush-like polymers and the zwitterionic brush-like polymers are capable of decreasing BSA and hMSC attachment compared to unmodified SF and existing “grafting to” methods, but that the zwitterionic compositions lead to greater reductions in attachment.

### V. ATR-FTIR and Contact Angle Measurements for Brush-Like Polymers on Silk Films Prepared by “Grafting From” Approach.

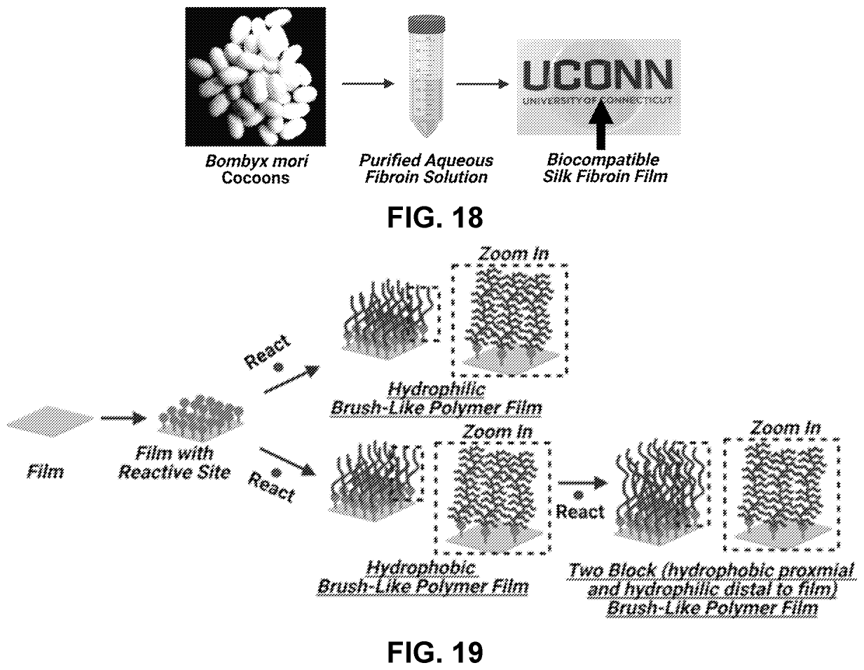

This experimental study used a “grafting from” approach by reacting monomers at the surface of silk films, which maximizes the number of polymer chains per film. Regenerated silk fibroin was selected as the base film material due its biocompatibility and biodegradability at an appropriate timescale, which allows the film to remain intact during drug delivery then ultimately degrade. Of note, regenerated silk fibroin is not the same silk format used in sutures, which are made of silk fibers coated with wax to prevent degradation. Regenerated silk fibroin contains no sericin, the protein that may induce an undesirable immune response. This experiment also removed sericin using a well-described technique, resulting in a pure fibroin solution that was cast into biocompatible, biodegradable films (FIG. 18).

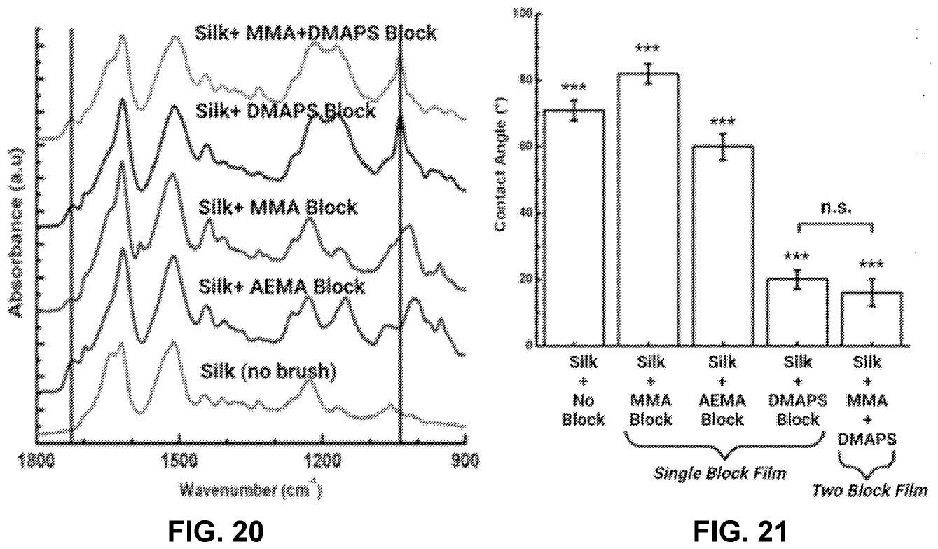

To ensure that the polymerization takes place on the surface, a molecule that initiates the polymerization must be covalently bound to the silk film. A variety of radical polymerizations were used to generate the brush-like polymers. To obtain brush-like polymers that may have different regions of compositions along the length of the brush, “living” polymerizations have been relied upon (e.g., reversible-addition fragmentation chain-transfer (RAFT), atom transfer radical polymerization (ATRP)), which allow the reaction to be paused to change the monomer (reactant) feed before resuming the reaction to continue the growth of the brush-like polymer. This technique permits the synthesis of brush-like polymers that contain regions of different monomer compositions connected together within a single brush. The studies focused on methacrylate and acrylate-based monomers, though other suitable monomers may be used. Monomers are selected for their side chains that have different degrees of hydrophobicity to generate brush-like polymers. In addition, a co-monomer containing a reactive group is used as the site where the therapeutic is attached via a tether molecule. Successful growth of hydrophilic brush-like polymers from the surface of silk films has been achieved using a type of chemistry called atom transfer radical polymerization (ATRP). Those brush-like polymers have lower water contact angles, cell attachment, and protein attachment than other silk surfaces designed to have low attachment. One key to the success of ATRP brush-like polymers was the identification of methods for enriching the silk surface with hydroxyl groups prior to polymerization. Building upon the successful hydroxylation, the reaction was used in combination with a different polymerization, RAFT (reversible-addition fragmentation chain-transfer), a “living” polymerization. First, hydrophobic, and hydrophilic monomers were each separately polymerized to generate brush-like polymers of a single composition (labeled as a “single block” for simplicity, where a “block” is a region of composition in the brush-like polymer) (FIG. 19) and then the silk-brush films were analyzed via ATR-FTIR (attenuated total reflectance Fourier transform infrared spectroscopy) to assess the success of the polymerization on the film surface. An amine-containing monomer and 2-aminoethylmethacrylate (AEMA) were also polymerized. All compositions showed a new peak (1729 cm⁻¹) via ATR-FTIR due the brush-like polymer's ester group (FIG. 20), thus confirming successful polymerization.

Next, vertical blocks of compositions were synthesized within the brush-like polymers. The hydrophobic monomer, MMA (methylmethacrylate) was polymerized from the silk surface. Upon reaction completion, the film was washed, dried, and analyzed by ATR-FTIR and contact angle measurements.

The functionalized film was placed back into the reactor and further polymerized with a hydrophilic zwitterionic monomer, DMAPS (2-(methacryloyloxy)ethyldimethyl-(3-sulfopropyl) ammonium hydroxide). The film was again washed and dried before being analyzed via ATR-FTIR and contact angle measurements. The analysis results (FIGS. 20-21) show a new peak in the FTIR and display the expected contact angle outcome: the surface becomes more hydrophobic when MMA brush-like polymers are generated on the silk, however, the surface switches to become more hydrophilic when the DMAPS block is added onto the existing MMA polymer. Brush-like polymers with vertical configurations of blocks that vary in their composition (e.g., hydrophobic/hydrophilic character, charge, functional groups) may thus be generated on the surface of silk films.

vi. Degradation and Cell Compatibility of Silk-Brush Films

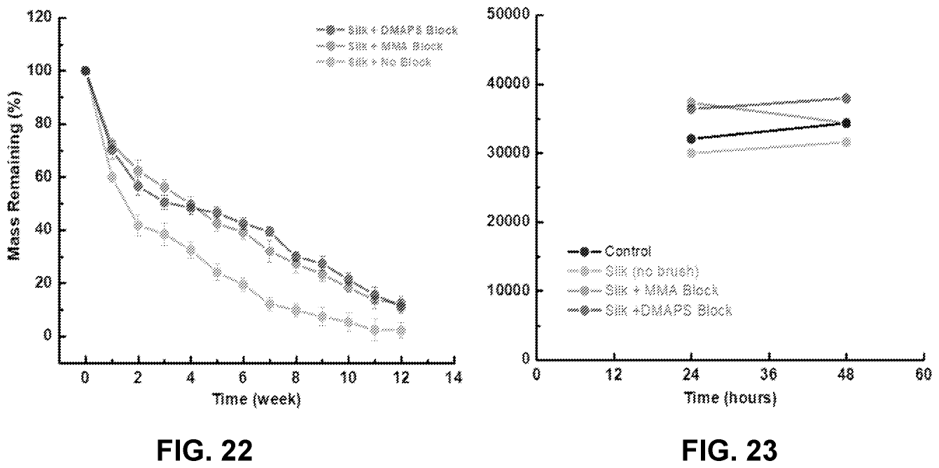

In vitro degradation shows the silk-brush films are stable in saline but will degrade over time in the presence of collagenase (FIG. 22). The films are thus expected to be intact as the active agent (e.g., therapeutic agent) is delivered, but ultimately degrade. Further, cell viability studies showed no significant impact on cellular growth from leachable components, confirming a non-toxic film (FIG. 23).

vii. Demonstration of Therapeutic Tethering and Release

As an exemplary therapeutic, bupivacaine was tethered to the silk-brush films. As proof of concept, bupivacaine loading was tested using 35 μg drug linked to three different films containing: hydrophobic blocks, hydrophilic blocks, and intermediate blocks.

Synthesis of Acryl Bupivacaine. Bupivacaine was modified with a hydrolyzable tether before conjugating to the polymer brushes. In this reaction, bupivacaine (BP, 500 mg, 1.74 mmol) was added to a round-bottom flask and dried by azeotropic vacuum distillation of water with toluene. The flask was purged with argon for 30 min before adding anhydrous THF (10 mL). A 60% NaH suspension in mineral oil (140 mg, 3.48 mmol) was slowly added while carefully venting the flask. The resulting mixture was allowed to stir at room temperature for 30 min. Acryloyl chloride (210 μL, 2.61 mmol) was added dropwise to the reaction flask equipped with a condenser and continued to react for 2 h at 80° C. in an oil bath. At the end of the reaction, the reaction was quenched by adding isopropyl alcohol (5 mL). The reaction product was concentrated under reduced pressure, and dissolved in dichloromethane (50 mL), and washed with dH₂O (100 mL) 3 times. The organic phase was collected, dried with MgSO₄, concentrated under reduced pressure, and stored in a desiccator.

Thiol Modification of Primary Amine Groups in AFMA Grafted SF films. The dried SF-Monomer+AEMA films were treated with 2-iminothiolane (Traut's Reagent, 45 mg, 0.45 mmol) in 1 ml Borate buffer. Additional water (5 ml) was added to the films and the reaction was stirred at room temperature for 2 h. After completion, the films were washed carefully with dH₂O and dried overnight at 60° C. The surface attached sulfhydryl groups were quantified using Ellman's reagent colorimetrically.

Covalent Conjugation of Acryl Bupivacaine to Polymer Grafts. The dried sulfhydryl modified SF films (0.0336 mmol of free SH) were placed in a glass scintillation vial. The pH of dH₂O (5 mL) was adjusted to 8.0 using triethanolamine before adding to a scintillation vial, and the solution was cooled using an ice-water bath. Acryl Bupivacaine (14 mg, 0.038 mmol) in dimethylformamide (DMF, 1.4 mL) was added to the reaction mixture. DMF (5 mL) was further added to maximize the solubility of acryl bupivacaine. The reaction was allowed to stir overnight at 4° C. At the end of the reaction, the films were thoroughly rinsed with dH₂O and dried overnight at 60° C.

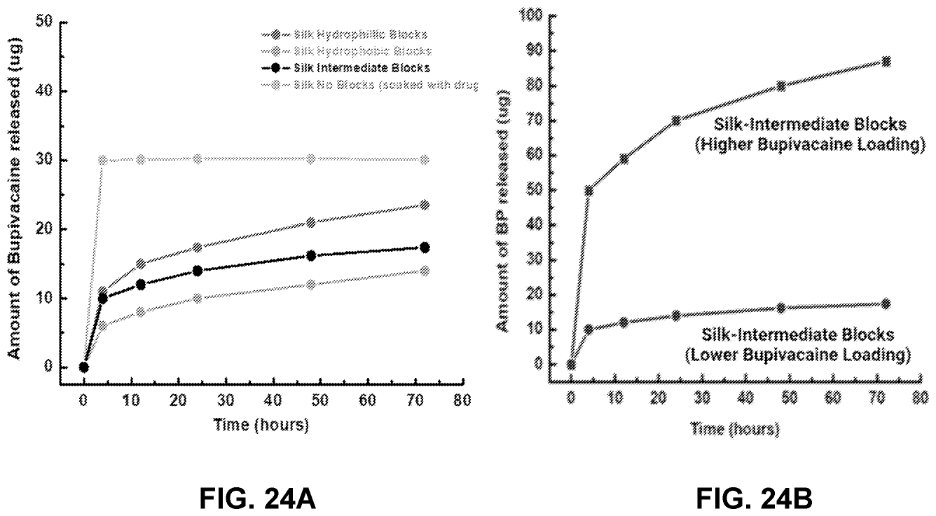

Successful release was shown after 24, 48, and 72 hours (FIG. 24A). Control films soaked (not covalently linked) with the same amount of drug released completely by 4 hours, demonstrating that the linking to blocks provides sustained release and that this may be tuned by modulating the block composition. Films were also loaded with higher levels of bupivacaine showing higher doses continually released over 72 hours, demonstrating clinically relevant amounts of drug may be delivered in the timeframe needed for post-operative pain (FIG. 24B). This data fully supports the ability to generate brush-like polymer surfaces of varied hydrophobicity (including vertical blocks of varying hydrophobicity) on silk film surfaces that release bupivacaine for at least 72 h. Furthermore, this data demonstrates that the presently disclosed silk-brush films exceed existing capabilities for sustained bupivacaine release.

viii. Longer-Term Cell Compatibility Studies

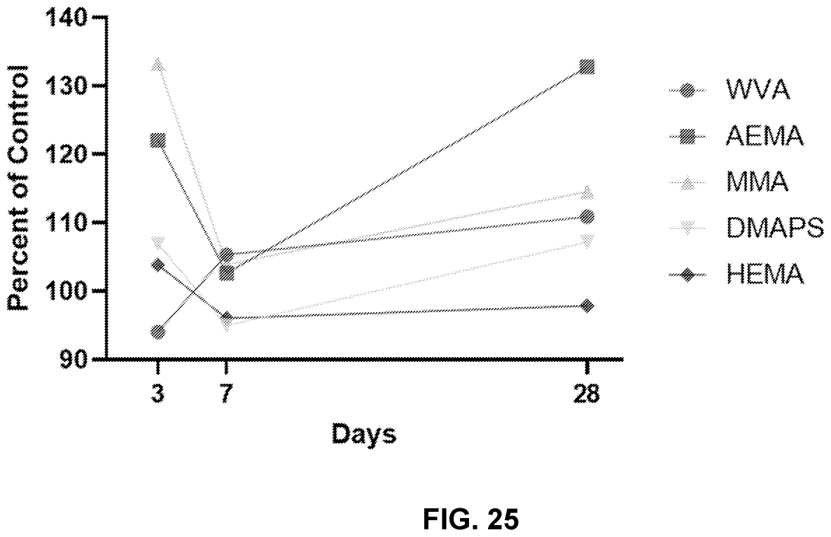

To further demonstrate cytocompatibility of brush-like polymer compositions, silk films containing brush-like polymers were soaked in media for up to 28 days. On the day prior to each time point, 3T3 fibroblast cells were plated at 35,000 cells per well in a 96-well plate. On day 3, 7, and 28 media was taken from the soaked film and replaced the spent media with incubating cells. After 24 hours the plate was measured with a CellTiter-Glo (Promega) ATP Assay. Measurements were compared as a percentage of the control with the control values being 100%. For all compositions tested, the cell viability was greater than 90% of the control value, demonstrating excellent cytocompatibility of the silk brush-like polymer films. (FIG. 25). As shown in FIG. 25, fluorescence signal indicating metabolic activity of 3T3 fibroblasts incubated with medium soaked with silk films containing brush-like polymers of varying compositions. All compositions tested showed greater than 90% of the metabolic activity relative to control, indicating excellent cytocompatibility of the brush-like polymer films.

For reasons of completeness, various aspects of the technology are set out in the following numbered clauses:

Clause 1. A composition comprising:
a silk protein film enriched with a plurality of hydroxyl groups; and a brush-like polymer attached to the silk protein film, the brush-like polymer comprising a plurality of repeating units, wherein each repeating unit comprises a backbone and a side chain.

Clause 2. The silk protein film of clause 1, wherein each side chain independently comprises a neutral pendant group, a positively charged pendant group, a negatively charged pendant group, or a combination thereof.

Clause 3. The silk protein film of clause 1 or 2, wherein each side chain independently comprises a zwitterionic pendant group, a hydrophilic pendant group, a hydrophobic pendant group, a pendant group attached to an active agent, or a combination thereof.

Clause 4. The silk protein film of any one of clauses 1-3, wherein the zwitterionic pendant group comprises a phosphodiester group, an ammonium group, a sulfonate group, a sulfobetaine group, a carboxybetaine group, a sulfopyridinium betaine group, a phosphorylcholine group, a cysteine group, a sulfobetaine siloxane group, a sulfobetaine acrylamide group, or a combination thereof.

Clause 5. The silk protein film of any one of clauses 1-4, wherein the hydrophilic pendant group comprises a polyethylene glycol (PEG) group, a hydroxyl group, an ether group, an ester group, a carboxylic acid group, a sulfonate, an aldehyde, a ketone, a thiol, an amine group, a nitro group, an imine group, a nitrile group, a thioether group, an amide group, or a combination thereof.

Clause 6. The silk protein film of clause 5, wherein the polyethylene glycol (PEG) group has a molecular weight of 44 g/mol to 2*10⁶ g/mol.

Clause 7. The silk protein film of any one of clauses 1-6, wherein the hydrophobic group comprises a C₁₋₂₀alkyl group, a C₁₋₂₀haloalkyl group, a C₃₋₆cycloalkyl group, a C₆₋₉aryl group, a 6- to 9-membered heteroaryl, or a combination thereof.

Clause 8. The silk protein film of any one of clauses 1-7, wherein each backbone is independently derived from monomers, the monomers comprising an acrylate monomer, a methacrylate monomer, a vinyl monomer, or a combination thereof.

Clause 9. The silk protein film of any one of clauses 1-8, wherein the brush-like polymer comprises:
a first block, the first block comprising a first plurality of repeating units; and
a second block, the second block comprising a second block of repeating units.

Clause 10. The silk protein film of any one of clauses 1-9, wherein the brush-like polymer is present on the silk protein film at a density of 1.0 chains/nm² to 3.0 chains/nm²

Clause 11. The silk protein film of any one of clauses 1-10, wherein the silk protein is silk fibroin.

Clause 12. The silk protein film of any one of clauses 1-11, wherein the silk protein comprises an amino acid sequence consisting of SEQ ID. No. 1.

Clause 13. The silk protein film of any one of clauses 1-12, wherein the active agent is a cell, a protein, a peptide, a nucleic acid, a nucleic acid analog, a nucleotide, an oligonucleotide, a peptide nucleic acid (PNA), an aptamer, an antibody, an antigen, an epitope, a hormone, a hormone antagonist, a growth factor, a recombinant growth factor, a cell attachment mediator, a cytokine, an enzyme, a small molecule, an antibiotic, an antimicrobial compound, a virus, an antiviral, or a combination thereof.

Clause 14. The silk protein film of any one of clauses 1-13, wherein the active agent comprises an alkylating agent, a platinum agent, an antimetabolite, a topoisomerase inhibitor, an antitumor antibiotic, an antimitotic agent, an aromatase inhibitor, a thymidylate synthase inhibitor, a DNA antagonist, a farnesyltransferase inhibitor, a pump inhibitor, a histone acetyltransferase inhibitor, a metalloproteinase inhibitor, a ribonucleoside reductase inhibitor, a TNF alpha agonist, a TNF alpha antagonist, an endothelin receptor antagonist, a retinoic acid receptor agonist, an immuno-modulator, a hormonal agent, an antihormonal agent, a photodynamic agent, or a tyrosine kinase inhibitor.

Clause 15. The silk protein film of any one of clauses 1-14, wherein the active agent comprises an aminoglycoside, bacitracin, a corbapenem, a cephalosporin, colistin, methenamine, a monobactam, a penicillin, polymyxin B, a quinolone, vancomycin, chloramphenicol, clindanyan, a macrolide, lincomyan, nitrofurantoin, sulfonamides, a tetracycline, trimethoprim, metronidazole, a fluoroquinolone, or ritampin.

Clause 16. The silk protein film of any one of clauses 1-15, wherein the active agent comprises edrophonium chloride, N-methylphysostigmine, neostigmine bromide, physostigmine sulfate, tacrine, tacrine, 1-hydroxy maleate, iodotubercidin, p-bromotetramiisole, 10-(alpha-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylamine, N′-monomethyl-L-arginine acetate, carbidopa, 3-hydroxybenzylhydrazine, hydralazine, clorgyline, deprenyl, hydroxylamine, iproniazid phosphate, 6-MeO-tetrahydro-9H-pyrido-indole, nialamide, pargyline, quinacrine, semicarbazide, tranylcypromine, N,N-diethylaminoethyl-2,2-diphenylvalerate hydrochloride, 3-isobutyl-1-methylxanthne, papaverine, indomethacind, 2-cyclooctyl-2-hydroxyethylamine hydrochloride, 2,3-dichloro-a-methylbenzylamine (DCMB), 8,9-dichloro-2,3,4,5-tetrahydro-1H-2-benzazepine hydrochloride, p-amino glutethimide, p-aminoglutethimide tartrate, 3-iodotyrosine, alpha-methyltyrosine, acetazolamide, dichlorphenamide, 6-hydroxy-2-benzothiazolesulfonamide, or allopurinol.

Clause 17. The silk protein film of any one of clauses 1-16, wherein the active agent comprises pyrilamine, chlorpheniramine, or tetrahydrazoline.

Clause 18. The silk protein film of any one of clauses 1-17, wherein the active agent comprises a corticosteroid, a nonsteroidal anti-inflammatory drug, acetaminophen, phenacetin, a gold salt, chloroquine, D-penicillamine, methotrexate colchicine, allopurinol, probenecid, or sulfinpyrazone.

Clause 19. The silk protein film of any one of clauses 1-18, wherein the active agent comprises mephenesin, methocarbomal, cyclobenzaprine hydrochloride, trihexylphenidyl hydrochloride, levodopa/carbidopa, or biperiden.

Clause 20. The silk protein film of any one of clauses 1-19, wherein the active agent comprises atropine, scopolamine, oxyphenonium, or papaverine.

Clause 21. The silk protein film of any one of clauses 1-20, wherein the active agent comprises aspirin, phenybutazone, idomethacin, sulindac, tolmetic, ibuprofen, piroxicam, fenamates, acetaminophen, phenacetin, morphine sulfate, codeine sulfate, meperidine, nalorphine, opioids, procaine, lidocaine, tetracaine, bupivacaine, ropivacaine, or dibucaine.

Clause 22. The silk protein film of any one of clauses 1-21, wherein the active agent comprises sodium fluorescein, rose bengal, methacholine, adrenaline, cocaine, atropine, alpha-chymotrypsin, hyaluronidase, betaxalol, pilocarpine, timolol, timolol salts, or combinations thereof.

Clause 23. The silk protein film of any one of clauses 1-22, wherein the active agent comprises imipramine, amitriptyline, nortriptyline, protriptyline, desipramine, amoxapine, doxepin, maprotiline, tranylcypromine, phenelzine, or isocarboxazide.

Clause 24. The silk protein film of any one of clauses 1-23, wherein the active agent comprises a platelet-derived growth factor (PDGP), a neutrophil-activating protein, a monocyte chemoattractant protein, a macrophage-inflammatory protein, a platelet factor, a platelet basic protein, a melanoma growth stimulating activity, an epidermal growth factor, a transforming growth factor alpha, fibroblast growth factor, platelet-derived endothelial cell growth factor, insulin-like growth factor, glial derived growth neurotrophic factor, ciliary neurotrophic factor, nerve growth factor, bone growth/cartilage-inducing factor alpha, growth/cartilage-inducing factor beta, a bone morphogenetic protein, an interleukin, an interferon, a hematopoietic factor, a tumor necrosis factor, or transforming growth factor beta.

Clause 25. The silk protein film of any one of clauses 1-24, wherein the active agent comprises an estrogen, an anti-estrogen, a progestin, an antiprogestin, an androgen, an anti-androgen, a thyroid hormone, or a pituitary hormone.

Clause 26. A method for preparing a brush-like silk protein film, the method comprising:
enriching a silk protein film with a plurality of hydroxyl groups;
functionalizing at least one of the hydroxyl groups with a reactive group;
synthesizing a brush-like polymer on at least one reactive group, the brush-like polymer comprising:

a plurality of repeating units, wherein each repeating unit comprises a backbone and a side chain.

Clause 27. The method of clause 26, wherein enriching the silk protein film comprises reacting the silk protein film with ethylene oxide.

Clause 28. The method of clause 26 or 27, wherein enriching the silk protein film comprises oxidizing the silk protein film.

Clause 29. The method of clause 28, wherein oxidizing the silk protein film comprises photocatalytic oxidation.

Clause 30. The method of any one of clauses 26-29, wherein functionalizing the silk protein film comprises reacting the silk protein film with an initiator.

Clause 31. The method of clause 30, wherein the initiator is α-bromoisobutyryl bromide (BIBB).

Clause 32. The method of any one of clauses 26-31, wherein synthesizing the brush-like polymer comprises polymerizing monomers.

Clause 33. The method of clause 32, wherein polymerizing the monomers comprises free radical polymerization, atom transfer radical polymerization of the monomers, or reversible addition-fragmentation chain transfer.

Clause 34. The method of clause 32 or 33, wherein the monomers comprise an acrylate monomer, a methylacrylate monomer, a vinyl monomer, or a combination thereof.

Clause 35. The method of any one of clauses 26-34, wherein each side chain independently comprises a neutral pendant group, a positively charged pendant group, a negatively charged pendant group, or a combination thereof.

Clause 36. The method of any one of clauses 26-35, wherein each side chain independently comprises a zwitterionic pendant group, a hydrophilic pendant group, a hydrophobic pendant group, a pendant group attached to an active agent, or a combination thereof.

Clause 37. The method of any one of clauses 26-36, wherein the zwitterionic pendant group comprises a phosphodiester group, an ammonium group, a sulfonate group, a sulfobetaine group, a carboxybetaine group, a sulfopyridinium betaine group, a phosphorylcholine group, a cysteine group, a sulfobetaine siloxane group, a sulfobetaine acrylamide group, or a combination thereof.

Clause 38. The method of any one of clauses 26-37, wherein the hydrophilic pendant group comprises a polyethylene glycol (PEG) group, a hydroxyl group, an ether group, an ester group, a carboxylic acid, a sulfonate, an aldehyde, a ketone, a thiol, an amine group, a nitro group, an imine group, a nitrile group, a thioether group, an amide group, or a combination thereof.

Clause 39. The method of clause 38, wherein the polyethylene glycol (PEG) group has a molecular weight of 44 g/mol to 2*10⁶ g/mol.

Clause 40. The method of any one of clauses 26-39, wherein the hydrophobic group comprises a C₁₋₂₀alkyl group, a C₁₋₂₀haloalkyl group, a C₃₋₄cycloalkyl group, a C₆₋₉aryl group, a 6- to 9-membered heteroaryl, or a combination thereof.

Clause 41. The method of any one of clauses 26-40 wherein the brush-like polymer comprises: a first block, the first block comprising a first plurality of repeating units; and a second block, the second block comprising a second block of repeating units.

Clause 42. The method of any one of clauses 26-41, wherein the brush-like polymer is present on the silk protein film at a density of 1.0 chains/nm² to 3.0 chains/nm².

Clause 43. The method of any one of clauses 26-42, wherein the silk protein is silk fibroin.

Clause 44. The method of any one of clauses 26-43, wherein the silk protein comprises an amino acid sequence consisting of SEQ ID. No. 1.

Clause 45. The method of any one of clauses 26-44, wherein the silk protein film is essentially free of sericin.

1. A composition comprising:
a silk protein film enriched with a plurality of hydroxyl groups; and
a brush-like polymer attached to the silk protein film, the brush-like polymer comprising a plurality of repeating units, wherein each repeating unit comprises a backbone and a side chain.

2. The silk protein film of claim 1, wherein each side chain independently comprises a neutral pendant group, a positively charged pendant group, a negatively charged pendant group, or a combination thereof.

3. The silk protein film of claim 1, wherein each side chain independently comprises a zwitterionic pendant group, a hydrophilic pendant group, a hydrophobic pendant group, a pendant group attached to an active agent, or a combination thereof.

4. The silk protein film of claim 3, wherein the zwitterionic pendant group comprises a phosphodiester group, an ammonium group, a sulfonate group, a sulfobetaine group, a carboxybetaine group, a sulfopyridinium betaine group, a phosphorylcholine group, a cysteine group, a sulfobetaine siloxane group, a sulfobetaine acrylamide group, or a combination thereof.

5. The silk protein film of claim 3, wherein the hydrophilic pendant group comprises a polyethylene glycol (PEG) group, a hydroxyl group, an ether group, an ester group, a carboxylic acid group, a sulfonate, an aldehyde, a ketone, a thiol, an amine group, a nitro group, an imine group, a nitrile group, a thioether group, an amide group, or a combination thereof.

6. The silk protein film of claim 5, wherein the polyethylene glycol (PEG) group has a molecular weight of 44 g/mol to 2*10⁶ g/mol.

7. The silk protein film of claim 3, wherein the hydrophobic group comprises a C₁₋₂₀alkyl group, a C₁₋₂₀haloalkyl group, a C₃₋₆cycloalkyl group, a C₆₋₉aryl group, a 6- to 9-membered heteroaryl, or a combination thereof.

8. The silk protein film of claim 1, wherein each backbone is independently derived from monomers, the monomers comprising an acrylate monomer, a methacrylate monomer, a vinyl monomer, or a combination thereof.

9. The silk protein film of claim 1, wherein the brush-like polymer comprises:
a first block, the first block comprising a first plurality of repeating units; and
a second block, the second block comprising a second block of repeating units.

10. The silk protein film of claim 1, wherein the brush-like polymer is present on the silk protein film at a density of 1.0 chains/nm² to 3.0 chains/nm².

11. The silk protein film of claim 1, wherein the silk protein is silk fibroin.

12. The silk protein film of claim 11, wherein the silk protein comprises an amino acid sequence consisting of SEQ ID. No. 1.

13. The silk protein film of claim 3, wherein the active agent is a cell, a protein, a peptide, a nucleic acid, a nucleic acid analog, a nucleotide, an oligonucleotide, a peptide nucleic acid (PNA), an aptamer, an antibody, an antigen, an epitope, a hormone, a hormone antagonist, a growth factor, a recombinant growth factor, a cell attachment mediator, a cytokine, an enzyme, a small molecule, an antibiotic, an antimicrobial compound, a virus, an antiviral, or a combination thereof.

14-25. (canceled)

26. A method for preparing a brush-like silk protein film, the method comprising:
enriching a silk protein film with a plurality of hydroxyl groups;
functionalizing at least one of the hydroxyl groups with a reactive group;
synthesizing a brush-like polymer on at least one reactive group, the brush-like polymer comprising:
a plurality of repeating units, wherein each repeating unit comprises a backbone and a side chain.

27. The method of claim 26, wherein enriching the silk protein film comprises reacting the silk protein film with ethylene oxide.

28. The method of claim 26, wherein enriching the silk protein film comprises oxidizing the silk protein film.

29. (canceled)

30. The method of claim 26, wherein functionalizing the silk protein film comprises reacting the silk protein film with an initiator.

31. (canceled)

32. The method of claim 26, wherein synthesizing the brush-like polymer comprises polymerizing monomers.

33. The method of claim 32, wherein polymerizing the monomers comprises free radical polymerization, atom transfer radical polymerization of the monomers, or reversible addition-fragmentation chain transfer.

34. The method of claim 26, wherein the monomers comprise an acrylate monomer, a methylacrylate monomer, a vinyl monomer, or a combination thereof.

35-45. (canceled)


SEQUENCE LISTING (1 sequence)

SEQ ID NO: 1 - protein, 5263 aa, Bombyx mori
MRVKTFVILCCALQYVAYTNANINDFDEDYFGSDVTVQSSNTTDEIIRDASGAVIEEQITTKKMQRKNKNHGILGKNEKMIKTFVITTDSDGNESIVEEDVLMKTLSDGTVAQSYVAADAGAYSQSGPYVSNSGYSTHQGYTSDFSTSAAVGAGAGAGAAAGSGAGAGAGYGAASGAGAGAGAGAGAGYGTGAGAGAGAGYGAGAGAGAGAGYGAGAGAGAGAGYGAGAGAGAGAGYGAGAGAGAGAGYGAGAGAGAGAGYGAASGAGAGAGYGQGVGSGAASGAGAGAGAGSAAGSGAGAGAGTGAGAGYGAGAGAGAGAGYGAASGTGAGYGAGAGAGYGGASGAGAGAGAGAGAGAGAGYGTGAGYGAGAGAGAGAGAGAGYGAGAGAGYGAGYGVGAGAGYGAGYGAGAGSGAASGAGSGAGAGSGAGAGSGAGAGSGAGAGSGAGAGSGAGAGSGAGAGSGAGAGSGTGAGSGAGAGYGAGAGAGYGAGAGSGAASGAGAGSGAGAGSGAGAGSGAGAGSGAGAGSGAGAGYGAGAGAGYGAGAGAGYGAGAGVGYGAGAGSGAASGAGAGSGAGAGSGAGAGSGAGAGSGAGAGSGAGAGSGAGAGSGAGAGSGAGAGSGAGVGYGAGVGAGYGAGYGAGAGAGYGAGAGSGAASGAGAGAGAGAGTGSSGFGPYVANGGYSRSDGYEYAWSSDFGTGSGAGAGSGAGAGSGAGAGSGAGAGSGAGAGSGAGAGYGAGVGVGYGAGYGAGAGAGYGAGAGSGAASGAGAGSGAGAGSGAGAGSGAGAGSGAGAGSGAGAGSGAGAGSGAGAGSGAGAGSGAGAGSGAGVGSGAGAGSGAGAGVGYGAGAGVGYGAGAGSGAASGAGAGSGAGAGSGAGAGSGAGAGSGAGAGSGAGAGSGAGAGSGAGAGSGAGAGSGAGVGYGAGVGAGYGAGYGAGAGAGYGAGAGSGAASGAGAGSGAGAGSGAGAGSGAGAGSGAGAGSGAGAGSGAGAGSGAGAGSGAGAGSGAGAGSGAGAGSGAGAGYGAGAGAGYGAGYGAGAGAGYGAGAGSGAASGAGSGAGAGSGAGAGAGSGAGAGSGAGAGSGAGAGSGAGAGSGAGAGSGAGAGYGAGVGAGYGAGYGAGAGAGYGAGAGSGAASGAGAGSGAGAGSGAGAGSGAGAGSGAGAGSGAGAGSGAGAGSGAGVGYGAGYGAGAGAGYGAGAGSGAASGAGAGAGAGAGTGSSGFGPYVAHGGYSGYEYAWSSESDFGTGSGAGAGSGAGAGSGAGAGSGAGAGSGAGYGAGVGAGYGAGYGAGAGAGYGAGAGSGAGSGAGAGSGAGAGSGAGAGSGAGAGSGAGAGSGAGAGSGAGAGSGAGAGSGAGAGYGAGYGAGAGAGYGAGAGSGAGSGAGAGSGAGAGSGAGAGSGAGAGSGAGAGSGAGAGSGAGAGSGAGAGYGAGVGAGYGAGYGAGAGAGYGAGAGSGAGSGAGAGSGAGAGSGAGAGSGAGVGSGAGAGSGAGAGSGAGAGSGAGAGYGAGYGAGAGAGYGAGAGSGAGSGAGAGSGAGAGSGAGAGSGAGAGSGAGAGSGAGAGSGAGAGSGAGVGYGAGVGAGYGAGYGAGAGAGYGAGAGSGAASGAGAGAGAGAGTGSSGFGPYVANGGYSGYEYAWSSESDFGTGSGAGAGSGAGAGSGAGAGSGAGAGSGAGAGYGAGYGAGAGAGYGAGAGSGAGSGAGAGSGAGAGSGAGAGSGAGAGSGAGAGSGAGAGSGAGAGSGAGSGSGAGAGSGAGAGSGAGAGYGAGVGAGYGVGYGAGAGAGYGAGAGSGAASGAGAGAGAGAGTGSSGFGPYVAHGGYSGYEYAWSSESDFGTGSGAGAGSGAGAGSGAGAGSGAGAGSGAGAGSGAGAGSGAGAGYGAGVGAGYGAAYGAGAGAGYGAGAGSGAASGAGAGSGAGAGSGAGAGSGAGAGSGAGAGSGAGAGSGAGAGSGAGAGSGAGAGSGAGAGSGAGAGYGAGAGAGYGAGAGSGAGSGAGAGSGAGAGSGAGAGSGAGAGSGAGAGSGAGSGSGAGAGSGAGAGSGAGAGYGAGVGAGYGAGYGAGAGAGYGAGAGSGAGSGAGAGSGAGAGYGAGAGAGYGAGYGAGAGAGYGAGAGTGAGSGAGAGSGAGAGSGAGAGSGAGAGSGAGAGSGAGAGSGAGSGSGAGAGSGAGAGSGAGAGSGAGAGSGAGAGSGAGAGYGAGAGAGYGAGYGAGAGAGYGAGAGSGAGSGAGAGSGAGAGSGAGAGSGAGAGYGAGYGAGAGSGAASGAGAGAGAGAGTGSSGFGPYVAHGGYSGYEYAWSSESDFGTGSGAGAGSGAGAGAGAGAGSGAGAGYGAGVGAGYGAGYGAGAGAGYGAGAGSGTGSGAGAGSGAGAGYGAGVGAGYGAGAGSGAAFGAGAGAGAGSGAGAGSGAGAGSGAGAGSGAGAGSGAGAGYGAGYGAGVGAGYGAGAGSGAASGAGAGSGAGAGSGAGAGSGAGAGSGAGAGSGAGAGYGAGVGAGYGAGYGAGAGAGYGAGAGSGAASGAGAGSGAGAGAGSGAGAGSGAGAGSGAGAGSGAGSGAGAGSGAGAGSGAGAGYGAGAGSGAASGAGAGAGAGAGTGSSGFGPYVANGGYSGYEYAWSSESDFGTGSGAGAGSGAGAGSGAGAGSGAGAGSGAGAGYGAGVGAGYGAGYGAGAGAGYGAGAGSGAGSGAGAGSGAGAGSGAGAGSGAGAGSGAGAGSGAGAGSGAGAGYGAGAGSGAASGAGAGSGAGAGSGAGAGSGAGAGSGAGAGSGAGAGSGAGAGYGAGVGAGYGVGYGAGAGAGYGAGAGSGAGSGAGAGSGAGAGSGAGAGSGAGAGSGAGSGAGAGSGAGAGSGAGAGSGAGSGAGAGSGAGAGYGVGYGAGAGAGYGAGAGSGAGSGAGAGSGAGAGSGAGAGSGAGSGAGAGSGAGAGSGAGAGSGAGAGYGAGVGAGYGVGYGAGAGAGYGAGAGSGAGSGAGAGSGAGAGSGAGAGSGAGAGSGAGAGSGAGAGSGAGAGSGAGSGAGAGSGAGAGSGAGAGSGAGAGSGAGSGAGAGSGAGAGSGAGAGSGAGAGYGAGVGAGYGVGYGAGVGAGYGAGAGSGAASGAGAGSGAGAGAGSGAGAGSGAGAGSGAGAGSGAGAGSGAGAGSGAGAGYGAGYGAGVGAGYGAGAGVGYGAGAGAGYGAGAGSGAASGAGAGAGSGAGAGTGAGAGSGAGAGYGAGAGSGAASGAGAGAGAGAGTGSSGFGPYVANGGYSGYEYAWSSESDFGTGSGAGAGSGAGAGSGAGAGSGAGAGSGAGAGYGAGVGAGYGAGAGSGAGSGAGAGSGAGAGSGAGAGSGAGAGSGAGAGYGAGAGSGTGSGAGAGSGAGAGSGAGAGSGAGAGSGAGAGSGAGAGSGVGAGYGVGYGAGAGAGYGVGYGAGAGAGYGAGAGSGTGSGAGAGSGAGAGSGAGAGSGAGAGSGAGAGSGAGAGSGAGAGYGAGVGAGYGVGYGAGAGAGYGAGAGSGAGSGAGAGSGAGAGSGAGAGSGAGAGSGAGSGAGAGSGAGAGSGAGAGSGAGSGAGAGSGAGAGYGVGYGAGAGAGYGAGAGSGAGSGAGAGSGAGAGSGAGAGSGAGSGAGAGSGAGAGSGAGAGSGAGAGYGAGVGAGYGVGYGAGAGAGYGAGAGSGAGSGAGAGSGAGAGSGAGAGSGAGAGSGAGAGSGAGAGSGAGAGSGAGSGAGAGSGAGAGSGAGAGSGAGAGYGAGVGAGYGVGYGAGAGAGYGAGAGSGAASGAGAGAGAGAGTGSSGFGPYVANGGYSGYEYAWSSESDFGTGSGAGAGSGAGAGSGAGAGYGAGYGAGVGAGYGAGAGVGYGAGAGAGYGAGAGSGAASGAGAGAGAGAGSGAGAGSGAGAGAGSGAGAGYGAGYGIGVGAGYGAGAGVGYGAGAGAGYGAGAGSGAASGAGAGSGAGAGSGAGAGSGAGAGSGAGAGSGAGAGSGAGAGYGAGYGAGVGAGYGAGAGVGYGAGAGAGYGAGAGSGAASGAGAGAGAGAGAGSGAGAGSGAGAGSGAGAGSGAGAGSGAGAGSGAGAGSGAGAGSGAGAGSGAGAGYGAGVGAGYGAGYGGAGAGYGAGAGSGAASGAGAGSGAGAGSGAGAGSGAGAGSGAGAGSGAGAGYGAGAGSGAASGAGAGAGAGAGTGSSGFGPYVNGGYSGYEYAWSSESDFGTGSGAGAGSGAGAGSGAGAGYGAGVGAGYGAGYGAGAGAGYGAGAGSGAASGAGAGSGAGAGSGAGAGSGAGAGSGAGSGAGAGSGAGAGSGAGAGSGAGAGSGAGAGSGAGAGYGAGVGAGYGAGYGAGAGAGYGAGAGSGAASGAGAGSGAGAGAGSGAGAGSGAGAGSGAGAGSGAGAGSGAGAGSGAGSGAGAGSGAGAGYGAGYGAGVGAGYGAGAGVGYGAGAGAGYGAGAGSGAASGAGAGSGSGAGSGAGAGSGAGAGSGAGAGAGSGAGAGSGAGAGSGAGAGYGAGYGAGAGSGAASGAGAGAGAGAGTGSSGFGPYVANGGYSGYEYAWSSESDFGTGSGAGAGSGAGAGSGAGAGYGAGVGAGYGAGYGAGAGAGYGAGAGSGAGSGAGAGSGAGAGSGAGAGSGAGAGSGAGAGSGAGAGSGAGAGSGAGAGYGAGYGAGAGAGYGAGAGVGYGAGAGAGYGAGAGSGAGSGAGAGSGSGAGAGSGSGAGSGAGAGSGAGAGSGAGAGSGAGAGSGAGAGSGAGAGSGAGAGYGAGYGIGVGAGYGAGAGVGYGAGAGAGYGAGAGSGAASGAGAGSGAGAGSGAGAGSGAGAGSGAGAGSGAGAGSGAGAGSGAGAGSGAGAGSGAGAGYGAGAGVGYGAGAGSGAASGAGAGSGAGAGSGAGAGSGAGAGSGAGAGSGAGAGSGAGAGSGAGSGAGAGSGAGAGYGAGYGAGVGAGYGAGAGYGAGYGVGAGAGYGAGAGSGAGSGAGAGSGAGAGSGAGAGSGAGAGSGAGAGSGAGSGAGAGYGAGAGAGYGAGAGAGYGAGAGSGAASGAGAGAGAGSGAGAGSGAGAGSGAGSGAGAGSGAGAGYGAGAGSGAASGAGAGSGAGAGAGAGAGAGSGAGAGSGAGAGYGAGAGSGAASGAGAGAGAGTGSSGFGPYVANGGYSRREGYEYAWSSKSDFETGSGAASGAGAGAGSGAGAGSGAGAGSGAGAGSGAGAGGSVSYGAGRGYGQGAGSAASSVSSASSRSYDYSRRNVRKNCGIPRRQLVVKFRALPCVNC